(19) United States
(12) Patent Application Publication
(10) Pub. No.: US 2025/0353990 A1
(43) Pub. Date: Nov. 20, 2025

(54) PROCESS FOR RECOVERING RAW MATERIALS FROM A POLYURETHANE MATERIAL

(71) Applicant: BASF SE, Ludwigshafen am Rhein (DE)

(72) Inventors: Andreas Thomas HAEDLER, Ludwigshafen am Rhein (DE); Mariana Sofia NOGUEIRA DUARTE, Ludwigshafen am Rhein (DE); Jens FERBITZ, Ludwigshafen am Rhein (DE); Ruth LOHWASSER, Ludwigshafen am Rhein (DE); Achim STAMMER, Ludwigshafen am Rhein (DE); Mona AL BATAL, Ludwigshafen am Rhein (DE)

(21) Appl. No.: 18/874,136

(22) PCT Filed: Jun. 1, 2023
(86) PCT No.: PCT/EP2023/064631

§ 371 Date: Dec. 12, 2024

(30) Foreign Application Priority Data
Jun. 14, 2022 (EP) 22178797.1

(51) Int. Cl. C08J 11/24; C07C 209/84

(52) U.S. Cl. CPC C08J 11/24; C07C 209/84

(57) ABSTRACT

A process for recovering an amine substance from a polyurethane material, preferably from an end-of-life (eol) polyurethane material, is provided, wherein the process comprises alcoholising a polyurethane material by contacting the polyurethane material with an alcoholising substance, wherein water is added in an amount so that a water content of a resulting mixture is from about 0.2 eq. to about 30 eq., based on the amount of cleavable bonds in the polyurethane material, wherein during alcoholising of the polyurethane material an amine substance and a polyol substance is formed, allowing the mixture to settle, wherein a phase, in particular a first phase, which is polyol substance rich, and a phase, in particular a second phase, which is alcoholising substance rich, are formed and wherein the process comprises a work-up of the mixture by purification of the amine substance, including distilling the phase, which is alcoholising substance rich, in particular the second phase, in a first distillation, in order to purify the amine substance, wherein the first distillation includes one or more distillation stages.

The present invention relates to a process for recovering raw materials from a polyurethane material. Exemplary raw materials are a polyol substance and/or an amine substance.

Furthermore, the present invention relates to a process for preparing polyurethane materials by reacting the polyol substance obtained by the process of the present invention with an isocyanate substance.

In addition, the present invention relates to a process for producing an isocyanate substance, preferably a toluene diisocyanate (TDI) substance, from an amine substance, preferably a toluene diamine (TDA)-based substance, obtained according to the process of the present invention.

Products containing polyurethane materials are widely used in industry and in everyday applications. Because of the tremendous and still increasing prevalence of polyurethane materials, there is a large amount of waste of polyurethane materials (e.g., from old mattresses or seating furniture or car seats). This waste of polyurethane materials should be used appropriately and as ecologically friendly as possible.

One way of such use of polyurethane materials is the recovery of raw materials from the polyurethane materials.

Apart from so-called mechanical recycling methods, which teach a physical comminution, chemical recycling processes are known.

From WO 2020/260387 A1 a method for recovering raw materials from polyurethane products is known, comprising the steps of (A) providing a polyurethane product that is based on an isocyanate component and a polyol component; (B) reacting the polyurethane product with a (mono- or polyvalent) alcohol in the presence of a catalyst, thereby obtaining a first product mixture; (C) obtaining the polyols from the first product mixture, comprising (C.I) mixing, without prior removal of any water that might be contained in the first product mixture, the first product mixture obtained in step (B) with an organic solvent that is not completely miscible with the alcohol used in step (B), and phase separation into a first alcohol phase and a first solvent phase; (C.II) processing the first solvent phase and obtaining polyols; and preferably (D) obtaining amines.

WO 2021/023889 A1 discloses a method for alcoholising and hydrolysing polyurethane materials made from at least one polyol compound and at least one toluene diisocyanate based compound, wherein the method comprises the following steps: contacting the polyurethane material with at least one alcoholising compound, thereby forming a reaction mixture (MO) and allowing the polyurethane material and the alcoholising compound to react in said reaction mixture (MO), thereby forming a mixture (M); allowing the mixture (M) to separate into at least an upper phase and a lower phase, wherein phase (A) and phase (B) are two immiscible phases; subjecting phase (B) to at least one hydrolysis step, thereby forming a phase (B1); wherein the at least one alcoholising compound is characterized by a melting point of lower than 200° C.; wherein the at least one alcoholising compound is characterized by a hydroxyl functionality of at least 2; and with the proviso that the at least one alcoholising compound is not glycerol.

However, there is the need for a process to recover raw materials having an optimized purity from polyurethane materials.

Concerning polyol substances recovered from polyurethane materials, the following has to be taken into consideration:
amine substances, such as toluene diamine, are often cancerogenic and thus need to be separated from the respective recovered polyol substance;
potassium ions as well as acid need to be removed since they render the respective recovered polyol substance less desirable for foaming application.

Regarding the recovery of amine substances from polyurethane materials, a method has to be developed according to which a trapping of the respective amine substance in carbamates is minimized. Furthermore, in order to obtain an amine that is suited for subsequent phosgenation, the amine substance has to be essentially free of solids, alkaline or acidic impurities as well as polyol residues. Amongst other process and product quality deficiencies, residual polyols will react with isocyanates during or after phosgenation and result in a reduction of NCO-content and can potentially lead to solids formation with an increased risk of equipment fouling.

In view of the above, it is an object of the present invention to provide a process to recover raw materials from polyurethane materials which is as simple as possible, and which allows to recover raw materials having an optimized quality.

This object is accomplished by the provision of a process according to claim 1.

In general, a polyurethane material comprises or consists of a structure which is formed by a polyaddition reaction of a (polyvalent) isocyanate (=the isocyanate substance of the polyurethane material) and a polyol (=the polyol substance of the polyurethane material). For example, a structure which is based on a diisocyanate O═C═N—R—N═C═O and a diol H—O—R′—O—H (wherein R and R′ denote organic radicals) can be as depicted as

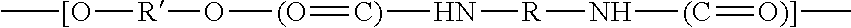

Many polyurethane materials, for example elastic and/or flexible polyurethane foam materials include urethane as well as urea bonds in a large quantity.

The process of the present invention preferably comprises an alcoholising step, preferably in combination with a hydrolysing step, in which a polyurethane material is contacted with an alcoholising substance. During alcoholising of the polyurethane material, a mixture containing a polyol substance and an amine substance is formed. According to a preferred aspect of the present invention, the polyurethane material is contacted with the alcoholising substance and water. Due to the addition of water or due to water being present in the original polyurethane material, a hydrolysis of the polyurethane material occurs. It is in this regard advantageous that any carbamates formed by transesterification are hydrolyzed to form amine and polyol. The presence of at least equimolar amounts of water can lead to about 100% amine liberation.

In particular for recovering an amine substance, it is beneficial if a polyurethane material is alcoholised by contacting the polyurethane material with an alcoholising substance, wherein water is added in an amount so that a water content of a resulting mixture is from about 0.2 eq. to about 30 eq. water, preferably to about 20 eq. water, in particular about 1 eq. to about 10 eq. water, in particular about 1.15 eq. to about 6 eq., for example about 1.3 eq. to about 4 eq., for example about 1.4 eq. to about 2 eq., based on the amount of cleavable bonds in the polyurethane material. During the alcoholising, i.e., alcoholysis, of the polyurethane material, an amine substance and a polyol substance are formed.

Eq. (i.e., equivalents) refers to equivalents per cleavable bond of the polyurethane material. Cleavable bonds are defined as urethane bonds and urea bonds within the polyurethane material and include segments in the polyurethane material in which segments are linked via allophanate units as well as linkages via biuret groups.

Basically, the amount of cleavable bonds is equivalent to the amount of the isocyanate group content in the original polyurethane material.

In case the polyurethane material already contains water, it is possible that no water needs to be added in order to adjust the water content of the mixture to be from about 0.2 eq. to about 30 eq. water, preferably to about 20 eq. water, in particular about 1 eq. to about 10 eq. water, in particular about 1.15 eq. to about 6 eq., for example about 1.3 eq. to about 4 eq., for example about 1.4 eq. to about 2 eq., based on the amount of cleavable bonds in the polyurethane material.

In case, the original water content of the polyurethane material is not in the described range, from about 0.2 eq. to about 30 eq. water, preferably to about 20 eq. water, in particular about 1 eq. to about 10 eq. water, in particular about 1.15 eq. to about 6 eq., for example about 1.3 eq. to about 4 eq., for example about 1.4 eq. to about 2 eq., based on the amount of cleavable bonds in the polyurethane material, can be added.

The water content of the mixture can also be adjusted to be about 0.2 wt.-% to about 35 wt.-%, preferably from about 0.2 wt.-% to about 10 wt.-%, in particular from about 2 wt.-% to about 8 wt.-%, for example from about 2.5 wt.-% to about 7 wt.-%, based on a total weight of the mixture.

Preferably, the process comprises allowing the mixture to settle, wherein a phase, in particular a first phase, which is polyol substance rich, and a phase, in a particular second phase, which is alcoholising substance rich, are formed.

Further, in particular for recovering the polyol substance, the process comprises preferably a work-up of the phase, which is polyol substance rich, in particular the first phase, by purification of the polyol substance. The purification comprises two or more of the following:
evaporation of the phase in one or more evaporators, in particular in a second distillation;
contacting the phase with an ion exchange material;
contacting the phase with one or more adsorbents.

After the work-up, the phase, which is polyol substance rich, in particular the first phase, preferably has an acid number of 0.1 mg KOH/g or less.

The acid number (corresponding to the acid value) is determined according to DIN EN ISO 4629-2, with minor changes. A mixture of iso-propanol/water 1:1 was used as solvent mixture, instead of toluene/ethanol 2:1. As a further change, NaOH/KOH was dissolved in methanol instead of ethanol.

In particular for recovering the amine substance, the process further comprises work-up of the mixture by purification of the amine substance, including the following:
distilling the phase, which is alcoholising substance rich, in particular the second phase, in a first distillation, in order to purify the amine substance.

The first distillation typically comprises one or more distillation stages. Preferably, the first distillation comprises at least two distillation stages. Preferred embodiments of the first distillation will be described in more detail below.

Preferably, the work-up of the mixture results in a partial or essentially complete release and/or recovery of the alcoholising substance.

Good results have been obtained for embodiments of the process, in which the amine substance comprises or consists of a toluene diamine (TDA)-based substance.

In the alternative to TDA-based amine substances, it is possible that the amine substance comprises or consists of a diamine or a polyamine of the diphenylmethane (MDA) series or of other amines, the corresponding isocyanates of which are typically used for the production of polyurethane materials.

In the context of the present invention, the term polyol substance encompasses all polyols known to the person skilled in the art in connection with polyurethane chemistry, such as, in particular, polyether polyols, polyester polyols, polyether ester polyols and polyether carbonate polyols. The term “one polyol substance” typically encompasses embodiments in which two or more different polyols have been used in the preparation of the polyurethane material. This also applies within a polyol class.

The phase, which is polyol substance rich, and the phase, which is alcoholising substance rich, can be identical. In these embodiments, the mixture is a one-phase system and/or a monophasic system.

In other particularly preferred embodiments of the process, the mixture is a multiphase system, preferably a biphasic system. In embodiments, in which the mixture is a biphasic system, the first phase is polyol substance rich and the second phase is alcoholising substance rich.

In multiphase systems, for example in biphasic systems, the different phases are immiscible at 25° C. For example, the first phase and the second phase are immiscible at 25° C.

“Polyol substance rich” preferably means that the respective phase contains at least about 46 wt.-%, preferably about 70 wt.-% or more, polyol substance, based on a total weight of the phase, in particular before work-up.

“Alcoholising substance rich” preferably means that the respective phase contains at least about 65 wt.-%, preferably about 70 wt.-% or more, alcoholising substance, based on a total weight of the phase, in particular before work-up.

The first phase preferably has a lower density than the second phase.

The polyurethane material preferably is a polyurethane foam, a polyurethane elastomer, a polyurethane adhesive, a polyurethane coating or a mixture thereof.

In particular in embodiments, in which the amine substance is recovered, it can be beneficial if an excess of water is removed from the mixture, before allowing the mixture to settle, preferably by evaporation of the excess of water. In particular, for evaporation of the excess of water, the mixture is heated and/or a vacuum is applied. For example, an excess of water is removed by using flash evaporation or applying vacuum to the already heated mixture.

For example, a water removal step is performed for about 120 minutes or less, in particular about 90 minutes or less, for example about 75 minutes or less, for example about 60 minutes or less.

Preferably, the water removal step is performed for about 10 minutes or more, in particular for about 30 minutes or more, for example for about 40 minutes or more.

In embodiments, in which flash evaporation is used for removing the water, the removal step, i.e., the flash evaporation, is performed for 15 minutes or less.

According to one aspect of the invention, solids are removed from the mixture before or after the mixture is allowed to settle, preferably by one or more of the following: filtration, centrifugation, decantation. The mentioned techniques are part of a solid-liquid-separation or form the solid-liquid-separation.

For example, after the alcoholysis or after an excess of water has been removed, the mixture is filtrated, in particular at a temperature from about 70° C. to about 200° C.

For filtration, preferably filter membranes having an average mesh size of about 30 μm or less are used. It can be beneficial to use a cascade of filters. For example, a first filter membrane of the cascade of filters has an average mesh size of about 250 μm to about 290 μm. A second filter membrane of the cascade of filters preferably has an average mesh size of about 50 μm to about 90 μm and, in particular, a third filter membrane of the cascade of filters has an average mesh size of 20 μm or less.

In the alternative or additionally, centrifugation is a preferred solid-liquid-separation method. For example, the mixture is centrifuged after alcoholysis or after an excess of water has been removed.

Preferably, the mixture is centrifuged at a temperature of 70° C. or more, in particular about 90° C. or more and/or about 160° C. or less, in particular about 140° C. or less. For example, the mixture is centrifuged at about 2500 rpm to about 14000 rpm, preferably at about 3000 rpm to about 6000 rpm.

Performing a solid-liquid-separation before a phase separation often leads to improved phase separation. In the solid-liquid-separation, solids which slow down or deteriorate the phase separation, are removed. For example, the formation of a mulm between two phases can be avoided or reduced. This mulm stabilizes the mixture and deteriorates phase separation.

It is also possible that more than one solid-liquid-separation steps are performed.

For example, the following solid-liquid-separation methods can be combined:
decantation and filtration; or
centrifugation and filtration.

It is possible that solid-liquid-separation steps are performed successively. Alternatively, one solid-liquid-separation step may be performed before allowing the mixture to settle and a further solid-liquid-separation step may be performed in the beginning of the work-up of the phase, which is polyol substance rich, in particular the first phase, or the work-up of the phase, which is alcoholising substance rich, in particular the second phase.

According to one aspect of the invention, it can be beneficial, if the one or more adsorbents are added before the solids are removed. For example, the one or more adsorbents can serve as a filtering aid.

According to one aspect of the invention, the mixture is contacted with one or more adsorbents before allowing the mixture to settle, wherein preferably the one or more adsorbents are selected from the group consisting of: activated carbon, silica, silicate, in particular alkali metal silicate and/or alkaline earth metal silicate, for example magnesium silicate, or a mixture of two or more thereof. Particularly preferred is the use of silicate, for example magnesium silicate and/or sodium silicate, as adsorbent before the phase separation.

According to one aspect of the invention, the phase, which is polyol substance rich, in particular the first phase, is purified by performing a solid-liquid separation, in which solids are removed, before the purification, wherein the solid-liquid-separation comprises of consists of one or more of the following: filtration, centrifugation, decantation.

According to a preferred embodiment, the phase, which is polyol substance rich, in particular the first phase, is filtrated, before the purification.

In particular the phase, which is polyol substance rich, in particular the first phase, is filtrated by a filter membrane having an average mesh size of about 20 μm or less.

The filtration is preferably performed by using a cascade of filters.

For example, a first filter membrane of the cascade of filters has an average mesh size of about 250 μm to about 290 μm. A second filter membrane of the cascade of filters preferably has an average mesh size of about 50 μm to about 90 μm and, in particular, a third filter membrane of the cascade of filters has an average mesh size of 20 μm or less.

According to one example, the phase, which is polyol substance rich, in particular the first phase, is allowed to sediment and an evolving supernatant is separated from solids by pumping and/or decanting the supernatant. The supernatant is afterwards treated further as the phase, which is polyol rich, in particular as the first phase.

In the alternative or additionally, centrifugation is a preferred solid-liquid-separation method. For example, the phase, which is polyol substance rich, in particular the first phase, is centrifuged before further purification.

Preferably, the phase, which is polyol substance rich, in particular the first phase, is centrifuged at a temperature of 70° C. or more, in particular about 90° C. or more and/or about 160° C. or less, in particular about 140° C. or less. For example, the phase, which is polyol substance rich, in particular the first phase, is centrifuged at about 2500 rpm to about 14000 rpm, preferably at about 3000 rpm to about 6000 rpm.

It can be beneficial, for example in embodiments, in which no solid-liquid-separation has been performed before, or directly after the mixture is allowed to settle, to remove solids from the phase, which is alcoholising substance rich, in particular the second phase. Preferred solid-liquid-separation methods are: filtration, centrifugation, decantation or a mixture of two or more thereof.

For an optimized yield of the release of raw materials, preferably the alcoholising step is performed in the presence of a catalyst, wherein preferably the catalyst is selected from the group consisting of alkali metal hydroxides, alkaline earth metal hydroxides, alkali metal salts of carboxylic acids, in particular acetates, alkaline earth metal salts of carboxylic acids, in particular acetates, Lewis acids, in particular dibutyltin dilaurate, organic amines, in particular imidazole or diethanolamine, organometallic compounds, in particular rare earth metal catalysts, for example titanium tetrabutoxide, or tin compounds, such as tin octoate.

As alkali metal hydroxides, the use of potassium hydroxide (KOH), sodium hydroxide (NaOH), or cesium hydroxide (CsOH) is particularly preferred.

In accordance with a particularly preferred embodiment, potassium hydroxide is used as a catalyst for the alcoholising reaction.

In accordance with another particularly preferred example, sodium hydroxide is used as catalyst.

In accordance with a preferred embodiment, the catalyst, for example potassium hydroxide or sodium hydroxide, is used in an amount of about 0.2 wt.-% or more and/or about 35 wt.-% or less, preferably about 5 wt.-% or less, in particular about 0.4 wt.-% or more and/or about 3.5 wt.-% or less, for example about 1 wt.-% or more and/or about 2 wt.-% or less, based on a total weight of the mixture.

Preferably, the catalyst, for example KOH or NaOH, is used in an amount of about 0.2 wt.-% or more and/or about 35 wt.-% or less, preferably about 6.5 wt.-% or less, in particular about 0.8 wt.-% or more and/or about 5 wt.-% or less, for example about 1 wt.-% or more and/or about 4 wt.-% or less, based on a total weight of the polyurethane material. In particular, the catalyst is used in an amount of about 1.5 wt.-% or more and/or about 3.5 wt.-% or less, based on the total weight of the polyurethane material.

During the step of allowing the mixture to settle, preferably a phase separation of the mixture into the first phase and the second phase occurs during and/or after settling.

In particular after the work-up, at the end of the process, the first phase has a potassium content of about 0.1 wt.-% or less, preferably about 0.01 wt.-% or less, for example about 0.005 wt.-% or less, based on the total weight of the phase. The above-mentioned thresholds are also valid for embodiments of the process, in which potassium hydroxide is used as catalyst for the alcoholising step.

Preferably, the potassium content is about 0.004 wt.-% or less, based on the total weight of the first phase.

For the determination of the potassium content, an automated wet chemical digestion is performed. Afterwards, the potassium content is determined by ICP/OES (Inductively Coupled Plasma/Optical Emission Spectrometry).

As alcoholising substance, preferably an alcohol is used.

For example, the alcoholising substance comprises or consists of one or more of the following: methanol, ethanol, ethylene glycol, diethylene glycol, propylene glycol, dipropylene glycol, methylene glycol, triethylene glycol, glycerol, 2-methyl-1,3-propanediol and mixtures of two or more thereof.

The inventors have observed that in embodiments, in which dipropylene glycol is used as alcoholising substance, monophasic mixture results (after settling).

For alcoholising the polyurethane material, preferably about 0.2 wt. parts or more and/or about 5 wt. parts or less alcoholising substance per wt. part polyurethane material, are used. Preferably, about 0.4 wt. parts or more and/or about 2 wt. parts or less alcoholising substance per wt. part polyurethane material, in particular about 0.6 wt. parts or more and/or about 1.5 wt. parts or less alcoholising substance per wt. part polyurethane material, for example about 0.8 wt. parts or more and/or about 1.2 wt. parts or less alcoholising substance per wt. part polyurethane material, are used.

According to a preferred embodiment, a phase separation is performed after the mixture has settled, wherein the phase, which is polyol substance rich, in particular the first phase, is separated from the phase, which is alcoholising substance rich, in particular the second phase.

Preferably, the phase separation is performed in a phase separation device. For improved phase separation, it can be beneficial to remove water included in the mixture, e.g., by flash evaporation. Water removal can also be done by applying vacuum to the mixture or simply distill the mixture at atmospheric pressure. In principle, phase separation might also be improved by addition of salt or by using specific internals in phase separation devices.

Optionally the phase separation is fostered by the addition of a halogenated or non-halogenated hydrocarbon, or a mixture of several halogenated or non-halogenated hydrocarbons, immiscible or only partially miscible with the alcoholising substance.

According to a further aspect, the phase separation is fostered by the addition of a glycol, for example diethylene glycol.

For a further improved purity of the polyol substance, it is beneficial if the work-up further comprises as a last step, a final distillation of the phase, which is polyol substance rich, in particular a final distillation of the first phase. As an alternative to the final distillation, it can be beneficial to apply vacuum while stirring and heating, for example 100° C. to 150° C., the phase, which is polyol substance rich.

The distillation as the last step of the work-up is in particular in the following referred to as third distillation. Preferably, the third distillation is performed by using one or more evaporators.

In accordance with a preferred embodiment, the one or more evaporators for the work-up of the phase, which is polyol substance rich, in particular the first phase, are selected from one or more of the following: thin film evaporator, short path evaporator, falling film evaporator, rotary evaporator.

The work-up of the phase, which is polyol substance rich, in particular the work-up of the first phase, comprises a first part in which one or more evaporators are used and a second part in which one or more adsorbents are used. However, as mentioned above, it can also be beneficial to use the one or more adsorbents before the one or more evaporators.

It is beneficial if the work-up of the phase, which is polyol substance rich, in particular the first phase, comprises a combination of evaporating the phase, which is polyol substance rich, in particular the first phase, in a thin film evaporator and afterwards evaporating the phase, which is polyol substance rich, in particular the first phase, in a short path evaporator.

The work-up of the phase, which is polyol substance rich, in particular the first phase, preferably comprises contacting the phase, which is polyol substance rich, in particular the first phase, with a cation exchange material or an anion exchange material or contacting the phase with a cation exchange material and an anion exchange material in sequence or simultaneously.

For example, an ion exchange material, a cation exchange material an anion exchange material or a mixture of both is used.

An anion exchange material can be used in a first ion exchange step and a cation material can be used in a second ion exchange step or vice versa. In the alternative, an anion exchange material and a cation exchange material can be used simultaneously.

As cation exchange material, a strongly or weakly acidic material can be used. The material may be a polymeric resin comprising or consisting of crosslinked polystyrene, polyacrylate or polymethacrylate polymers containing carboxylic or sulfonic acid groups. Strongly acidic materials containing sulfonic acid groups are preferred. Materials of this type include LEWATIT® K 2431, LEWATIT® K 2621 and LEWATIT® K 2629 obtainable from LANXESS Deutschland GmbH, Amberlyst® 15, Amberlyst® 35 and Amberlyst® 40 from DuPont™ and C150SH and C160SH from Purolite GmbH, Germany.

In another embodiment, weakly acidic materials containing carboxylic acid groups are used. Materials of this type include Amberlite® MAC-3 H from DuPont™ or LEWATIT® CNP 80 from LANXESS Deutschland GmbH.

As anion exchange material, a weakly or strongly basic material is used. The material may be a polymeric resin consisting of crosslinked polystyrene, polyacrylate or polymethacrylate polymers containing tertiary and/or quaternary amino groups. Strongly basic materials containing quaternary amino groups are preferred. Examples of such materials are Amberlite® HPR 9000 OH from DuPont™ or LEWATIT® S 6368 A, obtainable from LANXESS Deutschland GmbH. In another embodiment, weakly basic materials containing tertiary amino groups are used. Examples of such materials are LEWATIT® MP 62 from LANXESS Deutschland GmbH or Amberlyst® A22 from DuPont™.

According to a preferred embodiment, the work-up of the phase, which is polyol substance rich, in particular the first phase, comprises two steps of evaporating the phase, which is polyol substance rich, in particular the first phase, in a thin film evaporator.

Preferably, the work-up of the phase, which is polyol substance rich, in particular the first phase, comprises contacting the phase, which is polyol substance rich, in particular the first phase, with one or more adsorbents, preferably activated carbon, silica, silicate, in particular alkali metal silicate and/or alkaline earth metal silicate, for example magnesium silicate, or a mixture of two or more thereof.

The adsorbent may be a neutral polymeric adsorbent or activated carbon. A preferred adsorbent is activated carbon. The activated carbon may be in powder form or granulated form. A preferred activated carbon to be used as an adsorbent is a chemically activated carbon. The chemically activated carbon may be derived from a plant material and activated with phosphoric acid. Activated carbons of this type are C GRAN or CNSP 1240 from Norit Activated Carbon or Acticarbone® BGE or Acticarbone® BGX from Chemviron Carbon GmbH, Germany. Other types of activated carbon may also be used.

It can be beneficial, if about 2 wt.-% to about 10 wt.-%, preferably about 3 wt.-% to about 8 wt.-%, of the one or more adsorbents are contacted with the phase, which is polyol substance rich, in particular the first phase, based on a total weight of the mixture of the phase and the one or more adsorbents.

In embodiments, in which silicate, for example, magnesium silicate, is used as adsorbent before phase separation, it is typically sufficient if an ion exchange material and/or activated carbon is used for purification of the phase, which is polyol substance rich, in particular the first phase. In particular, a particulate material is used as adsorbent. For example, a particulate material having an average particle size of about 15 μm or more, in particular of about 25 μm or more, for example of about 35 μm or more, is used as adsorbent.

Preferably, a particulate material having an average particle size of about 2 mm or less is used.

For example, a particulate material having an average particle size of about 85 μm or less, in particular of about 75 μm or less, for example of about 65 μm or less, is used as adsorbent.

A preferred silicate to be used as an adsorbent is magnesium silicate or sodium silicate or a mixture thereof.

Preferably, the one or more adsorbents are used in an amount of about 0.2 wt.-% to about 20 wt.-%, for example about 0.3 wt.-% to about 5 wt.-%, based on the total weight of the first phase.

In particular a particulate adsorbent is used. According to one embodiment, a mixture of activated carbon and magnesium silicate powder is used.

As adsorbent, preferably an amorphous material is used having a porous internal structure and/or an increased activated surface compared to a non-porous material. Thus, impurities can adsorb on the surface of the adsorbent and be removed from the phase, which is polyol substance rich, in particular from the first phase.

As already mentioned, an alkali metal silicate or an alkaline earth metal silicate or a mixture of the same can be used. For example, a magnesium silicate can be used as adsorbent. Magnesium silicate is particularly suitable for removal of potassium ions, which is often used as a catalyst during alcoholising.

In the alternative or in addition to magnesium silicate, sodium silicate is considered to be a suitable adsorbent.

According to a preferred embodiment, after the work-up of the first phase, the first phase finally has a content of aromatic amines of about 0.03 wt.-% or less, preferably about 0.01 wt.-% or less, for example about 0.005 wt.-% or less, based on a total weight of the first phase. “After the work-up of the first phase” refers to a state after the process has been performed and when the first phase is ready for further use, e.g., as a polyol for polyurethane production.

This content of the aromatic amines in particular refers to embodiments, in which the mixture is biphasic and separates into the first phase, which is polyol substance rich, and the second phase, which is alcoholising substance rich.

The amount of the aromatic amines is determined by liquid chromatography techniques. For example, 0.01 g of sample to be analyzed (or 0.5 g in case of very low amounts of aromatic amines are present in the sample) is diluted to 20 ml acetonitrile and filtered over a filter having a mesh size of 0.2 μm. Afterwards, the resulting mixture is injected into a reversed phase (RP) high-pressure liquid chromatography (HPLC) with ultraviolet (UV) detection. Amine content is determined with two consecutive runs and quantified with a 5-point calibration using an external standard.

With the drastically decreased amount of aromatic amines, such as toluene diamine (TDA), the resulting polyol substance is particularly suitable for use as reagent for new polyurethane materials. In particular due to the carcinogenic properties of TDA, the amount of TDA in the polyol substance rich phase should be as small as possible.

The inventors have found that it is beneficial if the alcoholising step is performed at an inner temperature of about 120° C. or more, more preferred about 150° C. or more, in particular about 160° C. or more, for example about 170° C. or more, is used. The inner temperature during the alcoholysis is preferably set to about 270° C. or less, more preferred to about 250° C. or less, in particular about 220° C. or less, for example about 200° C. or less. The inner temperature refers to the temperature of the mixture.

In embodiments, in which additional water is added, the water can be added before heating the mixture, while the mixture is heated or after the mixture has reached the desired temperature.

In embodiments, in which water is added, an inner temperature of the mixture is preferably kept at about 150° C. to about 240° C., in particular at about 170° C. to about 220° C., and at a pressure of about 1 bar to about 60 bar, during and after water addition.

The water can be added in a constant process over the alcoholising step. In a preferred embodiment, the water is added in such way that the inner temperature of the mixture is kept constant. In particular in such way that the inner temperature is kept at about 150° C. or more, more preferably at 170° C. or more.

According to a preferred embodiment, the alcoholising step is performed under inert gas atmosphere, for example under nitrogen atmosphere. This can reduce discoloration effects. In the alternative, the alcoholising step is performed under air. Optionally a reflux condenser can be used.

It can be beneficial, if the mixture is degassed by using standard techniques before the alcoholysis is performed.

A reaction time under reflux and/or the reaction time for the alcoholising reaction is preferably set to be about 10 minutes or more, in particular about 20 minutes or more, for example about 30 minutes or more, for example about 40 minutes or more.

Preferably, the reaction time under reflux and/or the reaction time for the alcoholising reaction is preferably set to be about 200 minutes or less, in particular about 175 minutes or less, for example about 150 minutes or less, for example about 135 minutes or less.

As mentioned, it might for some embodiments be beneficial, if the alcoholising is performed at a pressure of up to about 60 bar, for example in a pressure reactor.

In embodiments, in which one or more evaporators are used during the work-up of the polyol substance rich phase, in particular the first phase, it is beneficial if one or more of the evaporators is a thin film evaporator or short path evaporator which is operated at about 180° C. to about 270° C. and/or at a pressure of about 0.1 mbar to about 30 mbar.

The use of a thin film evaporator facilitates mass transfer processes in the substance to be purified, for example, in the polyol substance.

After the work-up of the phase, which is polyol substance rich, in particular after the work-up of the first phase, the phase preferably has a content of the polyol substance of 97 wt.-% or more, in particular of 99 wt.-% or more, based on the total weight of the phase.

The amount of polyol substance is determined by GPC (Gel Permeation Chromatography). As eluent, tetrahydrofuran (THF) is used. THF with toluene as solvent is used as internal standard. As column material styrene-divinylbenzene (SDV) having a pore size of 1000 angstrom and a particle diameter of 5 μm is used. One precolumn of 5 cm and two columns of 30 cm are used. Refractive index (RI) and/or UV/Vis detectors are used. A calibration with polyethylene glycol (PEG) obtained from PSS Polymer Standards Service GmbH, 55120 Mainz, Germany, is performed. The polyol content was then determined using a 5-point calibration of the respective area integral compared to Lupranol® 2074 (a trifunctional polyether polyol and contains predominantly secondary hydroxyl groups) as external standard, obtained from BASF Polyurethanes GmbH, 49448 Lemförde, Germany.

With the process of the present invention, about 95% or more, preferably about 97% or more, of the polyol substance which is theoretically recoverable from the polyurethane material can be released. Thus, the yield of the polyol substance release is about 95% or more, preferably about 97% or more.

“Release of polyol substance” in particular describes the free-up and/or liberation of polyol from all urethane bonds present in the original polyurethane material.

According to the present invention, the yield of the polyol substance release is determined by dividing the amount of polyol substance in both layers quantified after the alcoholising step by the amount of polyol substance theoretically obtainable from the polyurethane material.

The theoretically obtainable amount of polyol substance is, according to the typical range of polyurethane slabstock foam formulations, about 50 wt.-% to about 70 wt.-% of the total weight of the polyurethane material.

As described before, according to the present invention, preferably polyurethane material having a water content is treated by the process. Preferred ranges for the water content have been described above. As also described above, the water content can either be originally present in the polyurethane material-either inherently or due to sorption processes- or additional water can be added.

In case additional water is added, preferably the water is added before and/or during an alcoholising reaction occurs. Preferably, after the alcoholising step, no further water is added.

In particular in order to obtain a biphasic mixture, the settling step is performed by adjusting a temperature of the mixture to room temperature (25° C.) to about 160° C., preferably to about 80° C. to about 120° C., and by keeping the mixture at this temperature until phase separation has occurred.

Regarding the embodiments in which water is present during the alcoholising step, a yield of the amine substance release after the alcoholising step is about 70% or higher, in particular of about 85% or higher.

“Release of amine substance” in particular describes the free-up and/or liberation of amine from all urethane bonds and urea bonds present in the original polyurethane material.

The yield of the release of the amine substance is defined as the sum of free aromatic amine substance in both layers quantified after the alcoholising step divided by the amount of aromatic amine substance theoretically obtainable from the polyurethane material.

The theoretically obtainable amount of amine substance is, according to the typical range of polyurethane slabstock foam formulations, used for the experiments, about 20 wt.-% to about 50 wt.-% of the total weight of the polyurethane material.

According to the present invention, the yield of the amine substance “recovery”—in contrast to “release”—is determined by dividing the amount of amine substance quantified after the work-up by the amount of amine substance theoretically obtainable from the polyurethane material.

Preferably the phase, which is polyol substance rich, in particular the first phase, is distilled in a second distillation, wherein a second distillate is obtained. The second distillate and/or each distillate that is obtained before the first distillation is performed, is preferably combined with the phase, which is alcoholising substance rich, in particular the second phase, before the first distillation.

Preferably, each alcoholising substance containing distillate is combined with the second phase before the first distillation.

For the second distillation, preferably one or more evaporators are used, for example one or more of the following: a thin film evaporator, a short path evaporator, a falling film evaporator, a rotary evaporator. In this regard, it is referred to the description above and below regarding the one or more evaporators.

Due to the combination of the second distillate with the phase, which is alcoholising substance rich, the second distillate can be distilled together with the phase, which is alcoholising substance rich, in the first distillation, in particular together with the second phase.

In embodiments, in which the mixture is a monophasic system, preferably the second distillation is performed before the first distillation, wherein both distillations are performed with the same phase, i.e., the mixture.

Preferably no separate hydrolysis of the phase, which is alcoholising substance rich, in particular the second phase, is performed. In particular no separate hydrolysis is performed after the alcoholysis of the polyurethane material has been completed.

The first distillation relates to the work-up and/or purification of the amine substance, for example toluene diamine (TDA).

It can be beneficial, if the phase, which is the alcoholising substance rich, in particular the second phase, is treated in a solid-liquid-separation before the first distillation is performed. A preferred solid liquid separation is extraction, in particular using a non-polar, aprotic solvent as extraction solvent, for example toluene, benzene or xylene. In a preferred embodiment, the extraction is performed with toluene. In order to reduce the solubility of the alcoholising substance in the organic solvent layer, additional water can be added.

In addition or in the alternative to extraction, one or more of the following techniques are used: filtration, centrifugation, decantation.

The extraction solvent can after phase separation be recycled to the extraction stage.

Preferably, the first distillation is performed at a temperature of about 130° C. to about 290° C. and at a pressure of about 1 mbar to about 1000 mbar, preferably at a temperature of about 130° C. to about 250° C. and at a pressure of about 1 mbar to about 400 mbar, in particular at a temperature of about 140° C. to about 250° C. and at a pressure of about 1 mbar to about 200 mbar.

According to a preferred embodiment, the first distillation comprises a first distillation stage, in which the alcoholising substance is removed by distillation, and a second distillation stage, in which the amine substance is purified by distillation.

Preferably, the first distillation is performed in one or more distillation columns, for example, one or more fractionating columns.

According to one aspect of the invention, a fractionating column is used for the first distillation, e.g., a fractionating column with 10 to 30 stages, more preferred 15 to 25 stages. According to another aspect of the invention a sump distillation with filtration unit in the evaporation loop is used.

In particular, the first distillation stage is performed at a temperature of about 130° C. to about 250° C. and at a pressure of about 10 mbar to about 1000 mbar.

The second distillation stage is, for example, performed at a temperature of about 140° C. to about 250° C. and at a pressure of about 1 mbar to about 200 mbar.

According to a further preferred embodiment, the first distillation further comprises a third distillation stage.

In embodiments having a third distillation stage, it can be beneficial, if
the first distillation stage is performed at a temperature of about 60° C. to about 270° C. and at a pressure of about 100 mbar to about 1000 mbar; and/or
the second distillation stage is performed at a temperature of about 130° C. to about 250° C. and at a pressure of about 10 mbar to about 500 mbar; and/or
the third distillation stage is performed at a temperature of about 140° C. to about 250° C. and at a pressure of about 1 mbar to about 200 mbar.

According to another preferred embodiment, the distillation stage of the first distillation in which the amine substance is purified can completely or partially be performed in a column of an existing amine purification column of an existing amine producing plant.

For example, the second distillation stage or the third distillation stage is performed in an existing amine purification column by feeding the material to be distilled, i.e., crude amine substance resulting from previous distillation stages, into an existing amine producing plant. This third distillation stage in an existing installation can be performed in one distillation column, e.g., a dividing wall column, or another fractionating column, or in a series of several distillation columns.

In embodiments, in which the amine substance is TDA, the crude amine substance (here: crude TDA) resulting from the respective distillation stage of the first distillation may be fed into a fractionating column of an existing TDA plant and may partially or completely form a reactant stream in a corresponding TDA purification.

For example, the crude TDA resulting from the recovery of TDA from a polyurethane material is fed into a tank, e.g., a storage tank, of the existing TDA plant and mixed with further crude TDA not resulting from the recovery from a polyurethane material. This crude TDA mixture is then preferably further purified by feeding the mixture into a fractionating column.

In the fractionating column of the TDA plant, preferably;
a low boiler fraction is drawn off via a head of the column; and/or
TDA is drawn off via a side draw in a withdrawal section of the fractionating column;
and/or
a high boiler section is drawn off via a sump of the fractionating column.

According to an alternative to drawing TDA off via a side draw, in particular if pre-purified TDA is distilled, TDA is drawn off via the head of the fractionating column of the TDA plant, after all DEG fractions have been drawn off, e.g., using a column in batch mode. Pre-purified TDA is, for example, TDA essentially without light boilers and essentially without vic-TDA.

Preferably, during the first distillation, water and/or alcoholising substance are separated from the phase, which is alcoholising substance rich, in a particular the second phase. The water and/or alcoholising substance obtained from the first distillation can be recycled into the phase separation step.

Furthermore, the invention relates to a process for producing an isocyanate substance, preferably a toluene diisocyanate (TDI) substance, from an amine substance obtained by a process according to the present invention, preferably a toluene diamine-based substance.

The advantages and/or features described in connection with the process for recovering an amine substance apply for the process for producing an isocyanate substance, too.

Preferably, the amine substance resulting from the recovery process is part or directly fed into a purification section of an amine producing plant (as described above), an amine storage tank or an isocyanate producing plant.

For the use of the amine substance in the isocyanate production, the amine substance needs to be essentially free of polyol substance, residual metals and silicon compounds.

Preferably the amine substance is phosgenated so that an isocyanate substance is formed.

In the context of the present invention, the term “isocyanate substance” encompasses all isocyanates known to the person skilled in the art in connection with polyurethane chemistry, such as, in particular, toluene diisocyanate (TDI; prepared from toluene diamine, TDA) or the di- and polyisocyanates of the diphenylmethane series (MDI; prepared from the di- and polyamines of the diphenylmethane series, MDA). The expression “isocyanate substance” also encompasses embodiments in which two or more different isocyanates (e.g. mixtures of MDI and TDI) have been used in the preparation of the polyurethane material. This also applies within one isocyanate class (that is to say, for example, also applies to various MDI types). The totality of all isocyanates used in the preparation of the polyurethane material is referred to as the isocyanate substance (of the polyurethane material). The isocyanate substance comprises at least one isocyanate.

Analogously, all of the polyols used in the preparation of the polyurethane material are referred to as polyol substance (of the polyurethane material). The polyol substance comprises at least one polyol. Preferably, the polyol substance comprises or consists of one or more polyols having a molecular weight of about 2500 to about 3500 g/mole, a nominal OH functionality of about 3 and having an OH number of about 48 mg KOH/g to about 56 mg KOH/g. An exemplary polyol substance may be a polyol obtainable from BASF under the product name Lupranol 2074.

The present invention further relates to a process for preparing a polyurethane material by reacting the polyol substance obtained by the process according to the present invention with an isocyanate substance, preferably the isocyanate substance obtained by a process of the present invention.

In embodiments, in which the polyol substance obtained by the process according to the present invention is used in a process for preparing a polyurethane material, up to 100% of virgin polyol can be replaced by the recovered polyol substance without significant drawback in product quality.

All advantages and/or features described in connection with the process for recovering a polyol substance apply for the process for preparing a polyurethane material, too.

Preferably, the polyurethane material is used in a mattress or a furniture part or a part of a car seat.

Further preferred features and/or advantages of the present invention form the subject matter of the following description and the graphical illustration of exemplary embodiments.

In the Figures:

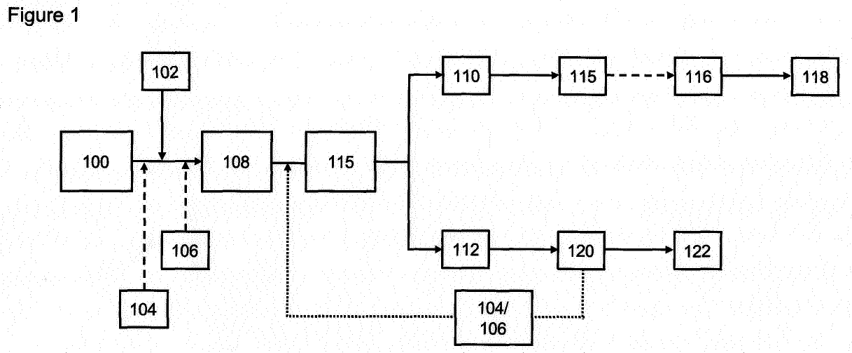

FIG. 1 schematically shows an embodiment of a process for recovering a polyol substance and/or an amine substance from an end-of-life (eol) polyurethane material, by performing a work-up of a mixture resulting from alcoholising the polyurethane material, wherein the mixture comprises a polyol substance rich phase and an alcoholising substance rich phase;

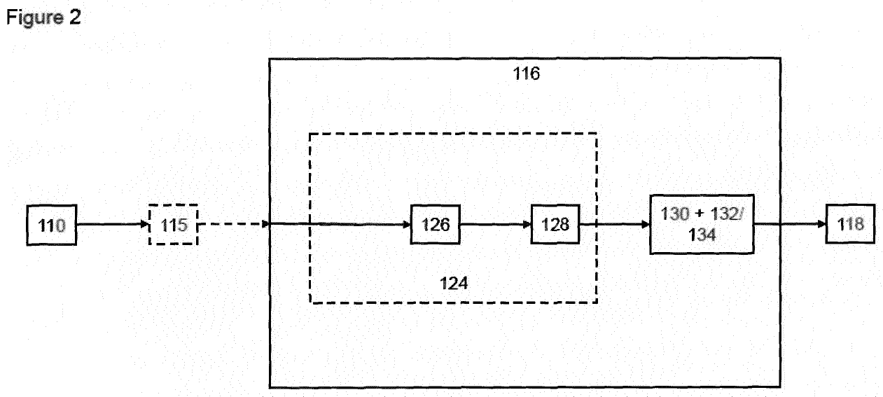

FIG. 2 schematically shows the work-up of the polyol substance rich first phase of the mixture of FIG. 1 in more detail; and

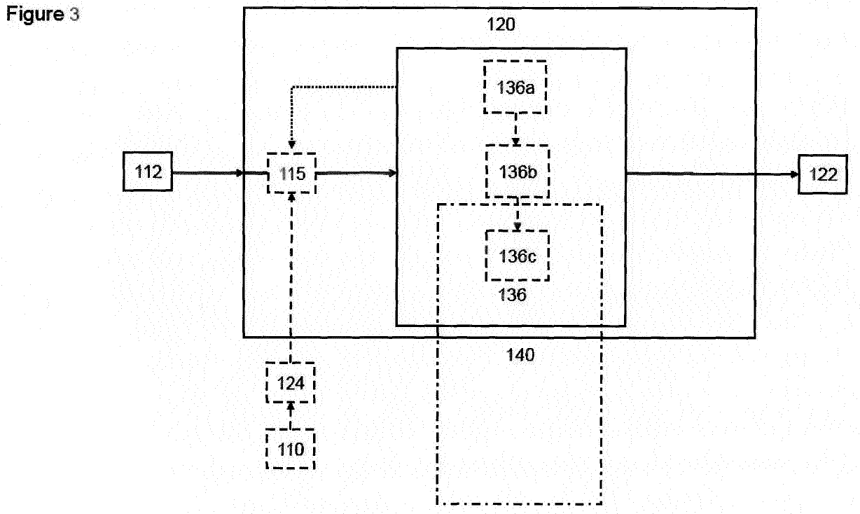

FIG. 3 schematically shows the work-up of the alcoholising substance rich second phase of the mixture of FIG. 1 in more detail.

Identical or functionally equivalent elements are designated in all figures with the same reference signs.

As stated above, the embodiments shown in the Figures and described in the following are exemplary and also other combinations of features are embodiments of the present invention.

FIG. 1 shows an exemplary embodiment of a process for recovering a polyol substance 118 and an amine substance 122 from an end-of-life (eol) polyurethane material 100. The eol polyurethane material 100 is presently a polyurethane foam, for example as obtained from mattresses or furniture or car seats.

It is to be understood that the steps of recovering the polyol substance 118 and the amine substance 122 can be performed simultaneously.

In the alternative, also only the polyol substance 118 or only the amine substance 122 can be recovered.

The recovery of the polyol substance 118 and the recovery of the amine substance 122 can also be performed spatially separate and/or in sequence.

According to the present invention, polyurethane materials 100 having a content of styrene acrylonitrile copolymer (SAN) of up to 15 wt.-%, based on a total weight of the polyurethane material 100, can be treated in the described process. The polyurethane material 100 having a SAN content in the mentioned range can be treated in the process according to the present invention although it has such a high content of SAN.

According to the presently described embodiment, the polyurethane material 100 is a polyurethane foam, which has been prepared by using an isocyanate substance in the form of toluene diisocyanate (TDI).

Accordingly, the amine substance 122 which is recovered is the corresponding amine, i.e., toluene diamine (TDA).

In embodiments, in which a polyurethane material 100 made of another isocyanate substance is used, the amine substance comprises or consists of the corresponding amine, respectively.

The polyurethane material 100 is combined with an alcoholising substance 102 for alcoholising. Thus, a mixture 108 is formed.

The alcoholising substance 102 comprises or consists of one or more of the following: methanol, ethanol, ethylene glycol, diethylene glycol, propylene glycol, dipropylene glycol, methylene glycol, triethylene glycol, glycerol, 2-methyl-1,3-propanediol and mixtures of two or more thereof.

For alcoholising the polyurethane material 100, preferably about 0.2 wt. parts or more and/or about 5 wt. parts or less alcoholising substance 102 per wt. part polyurethane material 100, are used. Preferably, about 0.4 wt. parts or more and/or about 2 wt. parts or less alcoholising substance 102 per wt. part polyurethane material 100, in particular about 0.6 wt. parts or more and/or about 1.5 wt. parts or less alcoholising substance 102 per wt. part polyurethane material 100, for example about 0.8 wt. parts or more and/or about 1.2 wt. parts or less alcoholising substance 102 per wt. part polyurethane material 100, are used. Further substances may be added for an optimized process.

Presently, a catalyst 104 in the form of potassium hydroxide (KOH) is used for the alcoholising of the polyurethane material 100.

In the alternative to potassium hydroxide, good results have been obtained if sodium hydroxide (NaOH) is used as a catalyst 104.

Preferably, the catalyst 104 is used in an amount of about 0.2 wt.-% or more, in particular of about 0.4 wt.-% or more, for example of about 1 wt.-% or more, based on a total weight of a resulting mixture 108.

In particular, the catalyst 104 is used in an amount of about 35 wt.-% or less, preferably about 5 wt.-% or less, for example of about 3.5 wt.-% or less, for example about 2 wt.-% or less, based on a total weight of the mixture.

It can be beneficial, if the catalyst 104 is used in an amount of about 0.2 wt.-% or more, preferably of about 0.8 wt.-% or more, in particular of about 1 wt.-% or more, based on the total weight of the polyurethane material 100.

Preferably, the catalyst 104 is used in an amount of about 35 wt.-% or less, preferably about 6.5 wt.-% or less, in particular of about 5 wt.-% or less, for example of about 4 wt.-% or less, based on the total weight of the polyurethane material 100.

In the alternative to using potassium hydroxide or sodium hydroxide, the catalyst 104 can be selected from the group consisting of other alkali metal hydroxides, alkaline earth metal hydroxides, alkali metal salts of carboxylic acids, in particular acetates, alkaline earth metal salts of carboxylic acids, in particular acetates, Lewis acids, in particular dibutyltin dilaurate, organic amines, in particular imidazole or diethanolamine, organometallic compounds, in particular rare earth metal catalysts, for example titanium tetrabutoxide, or tin compounds, such as tin octoate.

Furthermore, in order to optimize the release of the amine substance 122, presently water 106 is added in an amount so that a water content of a resulting mixture 108 is from about 0.2 wt.-% to about 20 wt.-%., in particular from about 3 wt.-% to about 8 wt.-% water, for example from about 4 wt.-% to about 7 wt.-% water, based on a total weight of the mixture 108.

Preferably, the water content is adjusted so that about 1 eq. to about 10 eq. water, in particular about 1.15 eq. to about 6 eq., for example about 1.3 eq. to about 4 eq., for example about 1.4 eq. to about 2 eq., is present in the mixture 108, based on the amount of cleavable bonds of the polyurethane material 100.

Whether additional water 106 is added is decided based on the water content of the polyurethane material 100 at a time when the process is started. This water content is referred to as “original water content” of the polyurethane material 100. The original water content includes previously adsorbed water as well as otherwise included water.

In particular, about 1 eq. to about 30 eq. water 106, preferably to about 10 eq. water 106, in particular about 1.15 eq. to about 6 eq., for example about 1.3 eq. to about 4 eq., for example about 1.4 eq. to about 2 eq., of additional water is added, based on the amount of cleavable bonds of the polyurethane material 100.

In embodiments, in which a polyurethane material 100 with a relatively high original water content is used, no or only little additional water 106 is added before or during alcoholising.

In embodiments, in which a polyurethane material 100 with an original water content is insufficient, 0.2 wt.-% to 30 wt.-% additional water 106 is added before or during alcoholising, based on the total weight of the mixture 108.

During alcoholising of the polyurethane material 100, the amine substance 118 and the polyol substance 122 are formed.

For the alcoholising (i.e., the alcoholysis) of the polyurethane material 100, the following reaction conditions have been found to be beneficial:
an inner temperature of 150° C. or more, for example about 170° C. or more, and/or about 240° C. or less, in particular about 220° C. or less, for example about 200° C. or less;
ambient pressure (e.g., normal pressure at about 1013.25 mbar) or a pressure of up to about 20 bar; and/or
inert gas atmosphere, e.g., nitrogen atmosphere, or air.

In the alternative to performing the alcoholysis in an inert gar atmosphere, the alcoholising can be performed under air. Optionally a reflux condenser can be used.

It can be beneficial, if the mixture is degassed by using standard techniques before the alcoholysis is performed.

A reaction time under reflux and/or the reaction time for the alcoholising reaction is preferably set to be about 10 minutes or more, in particular about 20 minutes or more, for example about 30 minutes or more, for example about 40 minutes or more.

Preferably, the reaction time under reflux and/or the reaction time for the alcoholising reaction is preferably set to be about 200 minutes or less, in particular about 175 minutes or less, for example about 150 minutes or less, for example about 135 minutes or less.

In particular in embodiments, in which the amine substance is recovered, it can be beneficial if an excess of water is removed from the mixture 108, before allowing the mixture to settle, preferably by evaporation of the excess of water. For example, for evaporation of the excess of water, the mixture 108 is heated and/or a vacuum is applied.

For example, an excess of water is removed by using flash evaporation or by applying vacuum to the already heated mixture 108.

For example, a water removal step is performed for about 120 minutes or less, in particular about 90 minutes or less, for example about 75 minutes or less, for example about 60 minutes or less.

Preferably the water removal step is performed for about 10 minutes or more, in particular for about 30 minutes or more, for example for about 40 minutes or more.

In embodiments, in which flash evaporation is used for removing the water, the removal step, i.e., the flash evaporation, is performed for 15 minutes or less.

According to one embodiment, the mixture 108 is contacted with one or more adsorbents 134 (not graphically shown) before further treatment. For example, the mixture is contacted with silicate, in particular with alkali silicate, e.g., sodium silicate, and/or earth alkaline silicate, e.g., magnesium silicate, before further treatment.

Preferably, the mixture is contacted with silicate, for example magnesium silicate, wherein a further mixture of the mixture 108 and the silicate is prepared. The further mixture preferably comprises 0.5 wt.-% to about 30 wt.-% silicate, in particular about 3 wt.-% to about 15 wt.-%, for example about 6 wt.-% to about 9 wt.-% silicate, based on the total weight of the further mixture. The further mixture is preferably heated to a temperature of about 60° C. to about 180° C., preferably about 80° C. to about 160° C., under vacuum, preferably at a pressure of about 20 mbar or less, in particular about 10 mbar or less, for example about 5 mbar or less.

For example, the mixture 108 is contacted with the silicate for about 100 minutes to about 140 minutes.

According to a preferred embodiment, solids are removed from the mixture 108 by a solid-liquid-separation 115. For example, the mixture 108 is filtrated. As an alternative or in addition to filtration, solids can be removed by centrifugation and/or decantation.

For centrifugation, preferably a centrifugation speed of about 2500 rpm to about 14000 rpm, preferably at about 3000 rpm to about 6000 rpm, is used. The mixture 108 is preferably centrifuged at a temperature of about 80° C. to about 160° C. The inventors have found that about 4 minutes to about 10 minutes is sufficient for a solid-liquid-separation 115.

It can be beneficial to transfer the mixture 108 to a phase separation device for phase separation.

Afterwards, the mixture 108 is allowed to settle, e.g., in a phase separation device. For improved phase separation, it can be beneficial to remove water included in the mixture 108, e.g., by flash evaporation, by applying vacuum to the mixture 108 or by distilling the mixture 108 at atmospheric pressure.

In addition or in the alternative to water removal, phase separation might also be improved by addition of salt or by using specific internals in phase separation devices.

Optionally the phase separation can be fostered by the addition of a halogenated or non-halogenated hydrocarbon, or a mixture of several halogenated or non-halogenated hydrocarbons, immiscible or only partially miscible with the alcoholising substance (not further described or graphically shown here).

In the alternative, a glycol, for example diethylene glycol, may be added to foster the phase separation (not graphically shown).

Presently, the mixture 108 separates into a first phase 110, which is polyol substance rich, and a second phase 112, which is alcoholising substance rich, during settling. The first phase 110 is the upper phase having a lower density than the second phase 112. The second phase 112 is the lower phase having a higher density than the first phase 110.

The settling step is performed at a temperature of the mixture 108 of about 25° C. (room temperature) to about 160° C., preferably to about 50° C. to about 150° C., for example to about 80° C. to about 120° C. The mixture 108 is kept at this temperature until phase separation has occurred.

Preferably no additional solvent is added for phase separation.

The first phase 110 and the second phase 112 are separated from each other.

For obtaining the polyol substance 118 in a high quality and with a minimum of impurities, such as potassium, aromatic amines, and a minimal acid number, a work-up 116 of the first phase 110 is performed. The work-up 116 of the first phase 110 is shown in more detail in FIG. 2.

In embodiments, in which the mixture 108 is a monophasic system, preferably the work-up is performed with the mixture 108 as a whole (not graphically shown).

According to one embodiment, the first phase 110 is contacted with one or more adsorbents 134 (not graphically shown) before further treatment. For example, the first phase 110 is contacted with silicate, in particular with alkali silicate, e.g., sodium silicate, and/or earth alkaline silicate, e.g., magnesium silicate, before further treatment.

As an alternative or in addition of a solid-liquid-separation 115 before the phase separation, the first phase 110 is presently treated within a solid-liquid-separation 115 before performing a work-up 116. In the described embodiment, the first phase 110 is filtrated, preferably by using one or more filter membranes having an average mesh size of about 10 μm to about 50 μm. In particular, the first phase 110 is filtrated by a filter membrane having an average mesh size of about 20 μm or less.

The filtration is preferably performed by using a cascade of filters, wherein for example a first filter membrane has an average mesh size of about 250 μm to about 290 μm, a second filter membrane has an average mesh size of about 50 μm to about 90 μm and a third filter membrane has an average mesh size of about 20 μm or less.

The mentioned filtration conditions are the preferred ones for each filtration used in the process (independent at which stage of the process).

In the alternative to the filtration, the first phase 110 can be purified in a centrifuge before further treatment and/or decantation is used. Concerning preferred centrifugation conditions, it is referred to the description below.

According to the presently described embodiment, the work-up 116 of the first phase 110 comprises purifying the first phase 110 by evaporating the same with one or more evaporators.

The evaporation of the first phase 110 in one or more evaporators is together referred to as second distillation 124.

Presently, two evaporators in the form of a thin film evaporator 126 followed by a short path evaporator 128 are used.

The thin film evaporator 126 is preferably operated at about 180° C. to about 270° C. at a pressure of about 6 mbar to about 20 mbar.

The short path evaporator 128 is preferably operated at about 180° C. to about 270° C. at a pressure of 0.1 mbar to about 10 mbar.

In addition or in the alternative to a thin film evaporator 126 and a short path evaporator 128 are a falling film evaporator and a rotary evaporator.

After the purification by evaporating the first phase 110, the first phase 110 is contacted with a cation exchange material 130 and afterwards with an anion exchange material 132 and/or the first phase 110 is contacted with one or more adsorbents 134, in particular with one or more particulate adsorbents 134.

Although presently shown in FIG. 2 in sequence, it is also possible, to contact the first phase 110 with a cation exchange material 130 and an anion exchange material 132 simultaneously or only use one of them.

As an alternative, the ion exchange material(s) can be used in-between two evaporator steps.

As cation exchange material 130, a strongly or weakly acidic material can be used. The material may be a polymeric resin comprising or consisting of crosslinked polystyrene, polyacrylate or polymethacrylate polymers containing carboxylic or sulfonic acid groups. Strongly acidic materials containing sulfonic acid groups are preferred. Materials of this type include LEWATIT® K 2431, LEWATIT® K 2621 and LEWATIT® K 2629 obtainable from LANXESS Deutschland GmbH, Amberlyst® 15, Amberlyst® 35 and Amberlyst® 40 from DuPont™ and C150SH and C160SH from Purolite GmbH, Germany.

In another embodiment, weakly acidic materials containing carboxylic acid groups are used. Materials of this type include Amberlite® MAC-3 H from DuPont™ or LEWATIT® CNP 80 from LANXESS Deutschland GmbH.

As anion exchange material 132, a weakly or strongly basic material is used. The material may be a polymeric resin consisting of crosslinked polystyrene, polyacrylate or polymethacrylate polymers containing tertiary and/or quaternary amino groups. Strongly basic materials containing quaternary amino groups are preferred. Examples of such materials are Amberlite® HPR 9000 OH from DuPont™ or LEWATIT® S 6368 A, obtainable from LANXESS Deutschland GmbH. In another embodiment, weakly basic materials containing tertiary amino groups are used. Examples of such materials are LEWATIT® MP 62 from LANXESS Deutschland GmbH or Amberlyst® A22 from DuPont™.

As adsorbent 134, one or more of the following materials is preferably used:
activated carbon; and/or
silicates, preferably alkali metal silicate or alkaline earth metal silicate or mixtures thereof, for example magnesium silicate and/or sodium silicate; and/or
silica.

In general, the adsorbent 134 may be a neutral polymeric adsorbent or activated carbon. A preferred adsorbent 134 is activated carbon. The activated carbon may be in powder form or granulated form. A preferred activated carbon to be used as an adsorbent is a chemically activated carbon. The chemically activated carbon may be derived from a plant material and activated with phosphoric acid. Activated carbons of this type are C GRAN or CNSP 1240 from Norit Activated Carbon or Acticarbone® BGE or Acticarbone® BGX from Chemviron Carbon GmbH, Germany. Other types of activated carbon may also be used.

Preferably, the one or more adsorbents 134 are used in an amount of about 0.2 wt.-% to about 20 wt.-%, in particular about 0.3 wt.-% to about 5 wt.-%, based on the total weight of the first phase 110. According to a particularly preferred embodiment, about 0.5 wt.-% to about 3 wt.-% adsorbent 134 is used, based on the total weight of a mixture between the respective phase 110 and adsorbent(s) 134.

In particular in embodiments, in which the first phase has already been contacted with one or more adsorbents 134, e.g., magnesium silicate, before the second distillation 124, only activated carbon is used as adsorbent after the second distillation 124.

It can also be beneficial to use the adsorbent 134, for example the silicate, e.g., magnesium silicate, before the mixture 108 is allowed to settle before the phase separation.

In embodiments, in which an adsorbent 134 has already been used before the work-up of the phase, which is polyol substance rich, in particular the first phase 110, typically only one adsorbent is used for the work-up. Preferably, this adsorbent 134 is an adsorbent, which has not yet been used earlier in the process. For example, activated carbon is used is used within the work-up of the phase, which is polyol substance rich, in particular the first phase 110.

Other combinations of the mentioned techniques of the work-up 116 of the first phase 110 are within the scope of the present invention and will be explained in detail in the detailed Examples below.

In particular after contacting the first phase 110 with the cation exchange material 130 and the anion exchange material 132 and/or the adsorbent 134, a further distillation (referred to a third distillation) may be performed to remove additional water from the first phase 110. The further distillation is not graphically shown. For the third distillation, the use of one or more evaporators, for example the use of a thin film evaporator, short path evaporator, falling film evaporator, rotary evaporator or mixtures of two of more thereof, is preferred.

As described, in the alternative to the use of one or more evaporators in the beginning of the work-up and optionally again in the end, it is also possible to firstly contact the first phase 110 with one or more adsorbents 134 and/or a cation exchange material 130 and an anion exchange material 132 and solely afterwards evaporate the first phase 110 (in a second distillation 124) (not graphically shown).

After the work-up 116, the first phase 110 preferably has one or more of the following properties:
a potassium content of about 0.1 wt.-% or less, preferably about 0.01 wt.-% or less, for example about 0.005 wt.-% or less, based on a total weight of the first phase 110;
a content of the alcoholising substance 102 of about 0.15 wt.-% or less, based on the total weight of the first phase 110 after the work-up 116;
1 a content of aromatic amines of about 0.03 wt.-% or less, preferably about 0.01 wt.-% or less, based on a total weight of the first phase 110 after the work-up 116;
a content of the polyol substance of about 97 wt.-% or more, in particular about 99 wt.-% or more, based on the total weight of the first phase 110 after the work-up 116;
an acid number of 0.1 mg KOH/g or less.

The acid number is determined based on DIN EN ISO 4629-2, with two minor changes. A mixture of iso-propanol/water 1:1 was used as solvent mixture, instead of toluene/ethanol 2:1. As a further change, NaOH/KOH was dissolved in methanol instead of ethanol. Acid number in the context of the present inventions relates to the total acid number of the respective substance, also referred to as the acid value.

For the determination of the potassium content, an automated wet chemical digestion is performed. Afterwards, the potassium content is determined by ICP/OES (Inductively Coupled Plasma/Optical Emission Spectrometry).

The content of aromatic amines is determined using a liquid chromatography. For example, 0.01 g of sample to be analyzed (or 0.5 g in case of very low amounts of aromatic amines are present in the sample) is diluted to 20 ml acetonitrile and filtered over a filter having a mesh size of 0.2 μm. Afterwards, the resulting mixture is injected into a reversed phase (RP) high-pressure liquid chromatography (HPLC) with ultraviolet (UV) detection.

Calibration solutions of the respective aromatic amines are prepared and calibration curves are taken using a 5-point calibration.

A reversed-phase column of silica particles, having a particle size of 3.5 μm and a pore size of 100 angstrom is used.

Response factors are determined by dividing the respective peak area by the calibration mass concentration.

Using the response factor, the concentration of the amine compound is determined.

OH numbers (corresponding to OH values) are determined based on DIN EN ISO 4629-2, but with 4-pyrrolidino-pyridin instead of DMAP being used.

The water content is determined by Karl-Fischer-titration. About 15 ml of anhydrous methanol is added to the titration flask and the Karl Fischer reagent is added to the determined endpoint. The substance is quickly added, stirred for one minute and titrated again with the Karl Fischer reagent to the end point.

The content of the alcoholising substance 102 (here: diethylene glycol (DEG)) is determined by gas chromatographic methods. About 0.4 g to 0.8 g of sample are diluted in 15 ml to 20 ml of a 1:1 mixture of ethyl acetate and methanol and undecane is added as internal standard. 5 to 6 drops of this mixture are reacted with 1 ml to 1.5 ml N-Methyl-N-trimethylsilyl-trifluoroacetamide (MSTFA) and the sample is injected to a gas chromatography (GC) equipped with a flame ionization detection (FID) device. The internal standard is used for quantification and two consecutive runs are performed.

The content of the polyol substance is determined by GPC (Gel Permeation Chromatography). In this regard, tetrahydrofuran is used as eluent. As solvent, a mixture of tetrahydrofuran and toluene is used as internal standard.

For the GPC, columns of styrene divinylbenzene having an average pore size of 1000 Å (angstrom) and an average particle diameter of 5 μm are used. One precolumn having a height of 5 cm and two columns having a height of 30 cm are used.

Afterwards, UV/Vis (ultraviolet/visible) spectroscopy is performed, wherein a calibration with PEG (polyethylene glycol) standards, obtainable from PSS GmbH, 55120 Mainz, Germany, is performed. Based on the resulting UV/Vis data, the content of the polyol substance is determined using a calibration of the area integral over Lupranol® 2074 (a trifunctional polyether polyol containing predominantly secondary hydroxyl groups). Lupranol® 2074 is commercially available from BASF Polyurethanes GmbH, 49448 Lemförde, Germany.

The potassium ion content is determined using inductively coupled plasma atomic emission spectroscopy. The sample is pretreated with acid prior to analysis.

As for the work-up 120 of the second phase 112, the distillate of the second distillation 124 performed during the work-up 116 of the first phase 110 is presently combined with the second phase 112 (cf. FIG. 3).

It is possible, to remove solids from the second phase 112 in a solid-liquid-separation 115 before further work-up 120. This is in particularly preferred in embodiments, in which no solid-liquid-separation 115 has been performed regarding the mixture 108.

The solid-liquid-separation 115 is preferably a filtration, a decantation, a centrifugation or a combination of two or more thereof.

Regarding further preferred conditions of the solid-liquid-separation 115, it is referred to the description above. These conditions are also valid for the removal of solids from the second phase 112.

Optionally the solid-liquid separation 115 can be an extraction. The extraction is performed with an aprotic, non-polar organic solvent, e.g., toluene, benzene, xylene or the like. In a preferred embodiment the extraction is performed with toluene. In order to reduce the solubility of the alcoholising substance 102 in the organic solvent layer, additional water might have to be added.

The obtained organic solvent rich layer is further treated in a first distillation step (referred to as first distillation 136). The aqueous layer, containing the alcoholising substance 102 and inorganic components is further treated in an evaporation step to recover the alcoholising substance 102, water and traces of the extraction solvent. The resulting stream is fed back to the phase separation step (as indicated in FIG. 1 by a dotted line), the remaining solids, containing inorganic salts and other impurities resulting from the additives used in preparation of the polyurethane material (e.g., silicones, catalysts, colors and the like) are discarded.

Consequently, a distillation is performed (referred to as first distillation 136) from which the amine substance 122 (presently the TDA) is obtained.

The first distillation 136 comprises one or more distillation stages 136a, 136b, 136c. In FIG. 3, three distillation stages as shown. According to the present invention, however, also only one or two distillation stages can be included into the process (as will be described in the following).

In particular, no separate hydrolysis of the second phase 112 is performed.

In a first embodiment, in which no extraction 115 has been performed, the first distillation 136 as a whole is performed at a temperature of about 130° C. to about 290° C. and at a pressure of about 1 mbar to about 1000 mbar, preferably at a temperature of about 130° C. to about 250° C. and at a pressure of about 1 mbar to about 400 mbar, in particular at a temperature of about 140° C. to about 250° C. and at a pressure of about 1 mbar to about 200 mbar.

Also in embodiments, in which no extraction has been performed, the first distillation 136 is performed in one or more distillation columns, for example a fractionating column.

According to a second embodiment, in which no extraction has been performed, the first distillation stage 136a can, for example, be performed at a temperature of about 130° C. to about 260° C. and at a pressure of about 500 mbar to about 1000 mbar in order to remove water. In a preferred variant, the first distillation stage 136a is performed at a sump temperature of 256° C. and a pressure of 1000 mbar and with a reflux ratio (in ton/ton) of 1.

In a second distillation stage 136b, the alcoholising substance 102 is removed, for example, by a distillation at a temperature of about 140° C. to about 250° C. and at a pressure of about 10 mbar to about 500 mbar, preferentially at about 10 mbar to 250 mbar and a sump temperature of about 140° C. to about 230° C. In case of diethylene glycol removal, this distillation sequence results in a residual diethylene glycol content of about 0 ppm to about 400 ppm at a reflux ratio of 1.

In a third distillation stage 136c, the amine substance is further purified, for example, by a distillation stage 136c at a temperature of about 140° C. to about 250° C. and a pressure of about 1 mbar to about 200 mbar.

In yet another embodiment, distillation stages 136a and 136b are performed in a combined distillation, removing water and alcoholising substance 102 in one distillation column.

The first distillation stage 136a can be performed at a pressure of about 50 mbar to about 500 mbar, preferentially about 50 mbar to about 200 mbar and a sump temperature of about 170° C. to about 250° C., preferably about 170° C. to about 220° C. The distillation to remove diethylene glycol and water is, for example, performed in sump distillation with filtration in the evaporation loop.

In a second distillation stage 136b, the remaining alcoholising substance 102 is removed in a packed or plate distillation column at temperatures of about 150° C. to about 250° C., preferably about 170° C. to about 220° C., at pressures of about 10 mbar to about 500 mbar, preferably about 10 mbar to about 200 mbar.

In a third distillation stage 136c, the amine substance is purified by distilling the amine substance at about 1 mbar to about 200 mbar, preferably about 1 mbar to 100 mbar, and at sump temperatures of about 140° C. to about 250° C.

According to an embodiment, in which an extraction of the second phase 112 with a non-polar, aprotic solvent has been performed before distillation 136, the first distillation 136 as a whole is preferably performed at a temperature of about 130° C. to about 290° C., preferably about 130° C. to about 250° C., in particular about 140° C. to about 250° C. The first distillation 136 as a whole is preferably performed at a pressure of about 1 mbar to about 1000 mbar, in particular about 1 mbar to about 400 mbar, for example about 1 mbar to about 200 mbar.

Preferably, the first distillation 136 is performed in one or more distillation columns, for example one or more fractionating columns.

The inventors have found that optimized results are achieved, if in a first distillation stage 136a of the first distillation 136, the non-polar aprotic solvent, for example toluene and residual alcoholising substance, for example diethylene glycol (DEG), is removed.

The first distillation stage 136a can, for example, be performed at a temperature of about 130° C. to about 250° C. and at a pressure of about 10 mbar to about 1000 mbar in order to remove toluene and DEG.

In the second distillation stage 136b, the amine substance is further purified, for example, by a distillation at a temperature of about 140° C. to about 250° C. and at a pressure of about 1 mbar to about 200 mbar.

The second distillation step can be done using the existing installation of an existing TDA plant.

Independent from the variant of the first distillation 136, the alcoholising substance and the water which have been removed by distillation, can be recycled into the phase separation step.

In embodiments, in which an extraction has been performed, the respective extraction solvent, e.g., the non-polar, aprotic solvent, can be recycled into the extraction step (as indicated by a dotted arrow in FIG. 3).

Independent from the variant according to which the first distillation 136 is performed, at least the final distillation stage, according to which the amine substance 122 is purified, can be performed in an existing fractionating column 140 of an existing amine producing plant. The fractionating column 140 of an existing amine producing plant is graphically indicated by a dotdashed line in FIG. 3.

In embodiments, in which the amine substance 122 is TDA, typically a crude TDA resulting from previous distillation stages 136a, 136b of the first distillation 136 is fed into a fractionating column 140 of an existing amine producing plant. The fractionating column 140 is preferably a divided wall column.

For example, the crude TDA is used as reactant stream and fed into a feed section of the divided wall column (not graphically shown). The crude TDA resulting from the first distillation stage 136a or the second distillation stage 136b may either form the reactant stream as a whole or be combined with more crude TDA resulting, e.g., from the hydrogenation of dinitrotuluene.

The crude TDA resulting from the first distillation or the crude TDA mixture is then preferably further purified by feeding the mixture into a fractionating column.

According to a preferred embodiment, a low boiler fraction is drawn off via the head of the column 140. Purified TDA is drawn off via a side draw in the withdrawal section of the column 140. A high boiler fraction is preferably drawn off via the sump of the column 140.

More details concerning typical TDA purification processes in existing TDA plants can be found in, e.g., EP1706370, EP1746083 and EP1864969. The content of the mentioned applications shall be fully incorporated into this application.

Due to and/or after the work-up 120 of the second phase 112, the second phase 112 has a content of aromatic amines, i.e., amine substance, of 6.5 wt.-% or more, in particular of 15 wt. % or more, based on the total weight of the second phase 112 after the work-up 120.

Preferably a yield of the polyol substance release of about 93% or more, in particular of about 94% or more, can be obtained.

In particular a yield of the amine substance release of about 70% or more, in particular of about 85% or more, can be obtained.

The yield of the polyol substance release is determined by dividing the amount of polyol substance quantified after the alcoholising step in both layers by the amount of polyol substance theoretically obtainable from the polyurethane material 100.

The yield of the amine substance release is determined by dividing the amount of amine substance quantified after the work-up 120 by the amount of amine substance theoretically obtainable from the polyurethane material 100.

With the process steps as described above, high-quality polyol substances 118 and/or high-quality amine substances 122 can be recovered from polyurethane materials 100, in particular from polyurethane foams.

The recovered polyol substance 118 is preferably used for preparing a polyurethane material, for example a polyurethane foam. Thus, the polyol substance 118 obtained by the process described above is reacted with an isocyanate substance (not graphically shown).

The produced polyurethane material can be used in mattresses or furniture parts or car seats.

The recovered amine substance 122, presently TDA, is preferably used to produce an isocyanate substance, presently TDI (not graphically shown).

For this, the TDA can be fed into a TDA plant (as graphically indicated in FIG. 3), into an TDA storage tank or into the phosgenation section of a TDI-plant.

For example, the amine substance 122 is phosgenated resulting in TDI.

The TDI produced can be used as isocyanate substance to produce polyurethane foam by reacting it with polyol substance 118 or any other suited polyol component.

In the following, preferred Examples are described in detail:

For all of the Examples described below, wt.-% are given with respect to the total weight of the respective mixture, phase etc.

For the Examples, a polyurethane foam material was used. For release yield, the following expectation values, i.e., theoretically obtainable amounts, were used:
65 wt.-% of the foam as polyol
23 wt.-% of the foam as toluene diamine.

The remaining amount is mainly constituted by additives and losses due to carbon dioxide formation during hydrolysis. It is expected that the polyurethane material comprises about 2 mole functional groups per kg (kilogram) polyurethane material. The functional groups in this regard are urethane groups and urea groups.

In the following, different Examples for lysis and phase separation experiments are described. Afterwards work-up Examples for the polyol substance and work-up Examples for the amine substance are described, starting off from one of the lysis and phase separation Examples. Within a recovery process as a whole (either the recovery of the polyol substance or the recovery of the amine substance or both), lyses and phase separation is combined with the respective work-up(s).

In the context of the present description and the accompanying claims, the term “lysis” means contacting a polyurethane material with an alcoholising substance and according to some Examples with water.

Afterwards, Examples 11 to 13 are provided, describing simulations of processes, including hydroglycolysis and the exemplary first distillations.

### EXAMPLES FOR LYSIS AND PHASE SEPARATION

It should be noted that for some Examples, no further work-up has been performed. For others, only a short work-up has been performed, which is described together with the lysis and phase separation.

### Example 1

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 200° C. for 2.5 h under nitrogen at ambient pressure with a reflux condenser.

Afterwards, the mixture is cooled to 80° C. and allowed to settle for 24 h while the temperature is kept at about 80° C. After 24 h, a phase separation into a first phase (upper layer, is polyol substance rich) and a second phase (lower layer, alcoholising substance rich) has occurred.

Directly after the phase separation, the first phase includes 80 wt.-% polyol, 10 wt.-% DEG, 3.07 wt.-% aromatic amines and 0.024 wt.-% potassium.

The second phase includes, directly after the phase separation, less than 5 wt.-% polyol, 72 wt.-% DEG, 8.56 wt. % aromatic amines and 1.6 wt.-% potassium.

During lysis and phase separation according to Example 1, 80% of the polyol and 57% of the amine could be released.

### Example 2

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 200° C. for 2.5 h under nitrogen at ambient pressure with a reflux condenser.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

Directly after the phase separation, the first phase includes 79 wt.-% polyol, 11 wt.-% DEG, 3.52 wt.-% aromatic amines and 0.029 wt.-% potassium.

The second phase includes, directly after the phase separation, less than 5 wt.-% polyol, 62 wt.-% DEG, 9.82 wt. % aromatic amines and 1.6 wt.-% potassium.

During lysis and phase separation according to Example 2, 90% of the polyol and 59% of the amine could be released.

### Example 3

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 190° C. for 5 h under nitrogen at ambient pressure with a reflux condenser.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

Directly after the phase separation, the first phase includes 76 wt.-% polyol, 12 wt.-% DEG, 3.32 wt.-% aromatic amines and 0.025 wt.-% potassium.

The second phase includes, directly after the phase separation, less than 5 wt.-% polyol, 68 wt.-% DEG, 8.87 wt. % aromatic amines and 1.5 wt.-% potassium.

During lysis and phase separation according to Example 3, 84% of the polyol and 56% of the amine could be released.

### Example 4.1

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 200° C. for 2.5 h under nitrogen at ambient pressure with a reflux condenser. Afterwards, the mixture is cooled to 80° C. and allowed to settle for 24 h while the temperature is kept at about 80° C. After 24 h, a phase separation into a first phase (upper layer, is polyol substance rich) and a second phase (lower layer, alcoholising substance rich) has occurred. Directly after the phase separation, the first phase includes 78 wt.-% polyol, 12 wt.-% DEG, 2.98 wt.-% aromatic amines and 0.022 wt.-% potassium.

The second phase includes, directly after the phase separation, less than 5 wt.-% polyol, 69 wt.-% DEG, 7.81 wt. % aromatic amines and 1.6 wt.-% potassium.

During lysis and phase separation according to Example 4.1, 95% of the polyol and 50% of the amine could be released.

### Example 4.2

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 200° C. for 2.5 h under nitrogen at ambient pressure with a reflux condenser.

Afterwards, the mixture is cooled to 140° C. and allowed to settle for 12 h while the temperature is kept at about 140° C. After 12 h, a phase separation into a first phase (upper layer, is polyol substance rich) and a second phase (lower layer, alcoholising substance rich) has occurred.

Directly after the phase separation, the first phase includes 78 wt.-% polyol, 12 wt.-% DEG, 2.98 wt.-% aromatic amines and 0.022 wt.-% potassium.

The second phase includes, directly after the phase separation, less than 5 wt.-% polyol, 69 wt.-% DEG, 7.81 wt. % aromatic amines and 1.6 wt.-% potassium.

During lysis and phase separation according to Example 4.2, 95% of the polyol and 50% of the amine could be released.

### Example 4.3

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 200° C. for 2.5 h under nitrogen at ambient pressure with a reflux condenser.

Afterwards, the mixture is cooled to 90° C. and centrifuged at this temperature for 5 minutes at 4000 rpm. After centrifugation, a phase separation into a first phase (upper layer, is polyol substance rich) and a second phase (lower layer, alcoholising substance rich) has occurred.

Directly after the phase separation, the first phase includes 78 wt.-% polyol, 12 wt.-% DEG, 2.98 wt.-% aromatic amines and 0.022 wt.-% potassium.

The second phase includes, directly after the phase separation, less than 5 wt.-% polyol, 69 wt.-% DEG, 7.81 wt. % aromatic amines and 1.6 wt.-% potassium.

### Example 4.4

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% potassium hydroxide as catalyst is formed (without additional water). The mixture is stirred at 200° C. for 2.5 h under nitrogen at ambient pressure with a reflux condenser.

Afterwards, the mixture is cooled to 120° C. and centrifuged at this temperature for 5 minutes at 4000 rpm. After centrifugation, a phase separation into a first phase (upper layer, is polyol substance rich) and a second phase (lower layer, alcoholising substance rich) has occurred.

Also a centrifugation at 140° C. for 5 minutes at 4000 rpm led to a successful phase separation.

Directly after the phase separation, the first phase includes 78 wt.-% polyol, 12 wt.-% DEG, 2.98 wt.-% aromatic amines and 0.022 wt.-% potassium.

The second phase includes, directly after the phase separation, less than 5 wt.-% polyol, 69 wt.-% DEG, 7.81 wt. % aromatic amines and 1.6 wt.-% potassium.

During lysis and phase separation according to Example 4.4, 95% of the polyol and 50% of the amine could be released.

### Example 5

Eol mattresses are sorted such that eol polyurethane foam is obtained.

Afterwards, a mixture of 48 wt.-% of eol polyurethane foam, 48 wt.-% diethylene glycol (DEG), 1.0 wt.-% potassium hydroxide as catalyst and 3.0 wt.-% water is formed. The mixture is stirred at 170° C. for 2 h under nitrogen at ambient pressure with a reflux condenser.

After the mixture has reacted for 1 h using a reflux condenser, a distillation is performed for 60 minutes at about 170° C.-190° C. in order to remove the excess of water partially.

Afterwards, the mixture is cooled to 80° C. and allowed to settle for 15.5 h while the temperature is kept at about 80° C. After 15.5 h, a phase separation into a first phase (upper layer, is polyol substance rich) and a second phase (lower layer, alcoholising substance rich) has occurred.

Directly after the phase separation, the first phase includes 81 wt.-% polyol, 13.1 wt.-% DEG, 5.7 wt.-% aromatic amines and less than 0.021 wt.-% potassium.

The second phase includes, directly after the phase separation, less than 5 wt.-% polyol, 75.4 wt.-% DEG, 13.8 wt. % aromatic amines and 1.2 wt.-% potassium.

During lysis and phase separation according to Example 5, more than 95% of the polyol and 86% of the amine could be released.

### Example 6

Eol mattresses are sorted such that eol polyurethane foam is obtained.

Afterwards, a mixture of 49.25 wt.-% of eol polyurethane foam, 49.25 wt.-% diethylene glycol (DEG) and 1.5 wt.-% sodium hydroxide as catalyst is formed (without additional water). The mixture is stirred at 200° C. for 2.5 h under nitrogen at ambient pressure with a reflux condenser.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

Directly after the phase separation the phases were filtrated, and the composition of the filtrates were determined. Accordingly, the first phase includes 79 wt.-% polyol, 14 wt.-% DEG, 3.91 wt.-% aromatic amines and less than 3 wt.-% potassium.

The second phase includes, directly after the phase separation, less than 5 wt.-% polyol, 64 wt.-% DEG, 8.04 wt. % aromatic amines and 0.1 wt.-% potassium.

During lysis and phase separation according to Example 6, 95% of the polyol and 46% of the amine could be released.

Example 6 illustrates that sodium hydroxide can be used as a catalyst.

### Example 7

Eol polyurethane foam containing 15 wt.-% styrene-acrylonitrile resin (SAN) obtained from eol mattresses is provided.

Afterwards, a mixture of 49.50 wt.-% of this eol polyurethane foam, 41.25 wt.-% diethylene glycol (DEG), 1.5 wt.-% potassium hydroxide as catalyst and 7.5 wt.-% additional water is formed. The additional water is added after the remaining components have been heated to 200° C. Upon the addition of the additional water, the temperature of the mixture is reduced to 140° C. The mixture is then stirred at 140° C. for 2 h under nitrogen at ambient pressure with a reflux condenser.

After the mixture has reacted for 2 h using a reflux condenser, a distillation is performed for 45 minutes in order to remove the excess of water.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

The first layer is filtrated using a filter element having a pore size of about 20 μm.

After the filtration, the first phase includes 59 wt.-% polyol, 19 wt.-% DEG, 8.40 wt.-% aromatic amines, 0.044 wt.-% potassium and 0.4 wt.-% water.

The second phase includes, directly after the phase separation, less than 5 wt.-% polyol, 51 wt.-% DEG, 13.81 wt. % aromatic amines and 0.1 wt.-% potassium.

During lysis and phase separation according to Example 7, 93% of the polyol and 80% of the amine could be released.

### Example 8

A model polyurethane foam comprising polyol (65 wt.-% of the foam) and toluene diamine (23 wt.-% of the foam) was used as eol polyurethane foam for this Example.

A mixture of 29.5 wt.-% eol polyurethane foam, 60.0 wt.-% diethylene glycol (DEG), 0.5 wt.-% potassium hydroxide as catalyst and 10 wt.-% additional water is formed. The additional water is added after the remaining components have been heated to 220° C. Upon the addition of the additional water, the temperature of the mixture is reduced to 120° C. The mixture is then stirred at 120° C. for 2.5 h under nitrogen at ambient pressure with a reflux condenser.

After the mixture has reacted for 2.5 h using a reflux condenser, a distillation is performed for 60 minutes in order to remove the excess of water.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

The first layer is filtrated using a filter element having a pore size of about 20 μm.

After the filtration, the first phase includes 79 wt.-% polyol, 16 wt.-% DEG, 3.98 wt.-% aromatic amines, 0.0009 wt.-% potassium and 0.3 wt.-% water.

The second phase includes, directly after the phase separation, less than 5 wt.-% polyol, 77 wt.-% DEG, 9.24 wt. % aromatic amines and 0.53 wt.-% potassium.

During lysis and phase separation according to Example 8, 90% of the polyol and 91% of the amine could be released.

### Example 9

A model polyurethane foam comprising polyol (65 wt.-% of the foam) and toluene diamine (23 wt.-% of the foam) was used as eol polyurethane foam for this Example.

A mixture of 46.25 wt.-% eol polyurethane foam, 47.5 wt.-% diethylene glycol (DEG), 1.25 wt.-% potassium hydroxide as catalyst and 5 wt.-% additional water is formed. The additional water is added after the remaining components have been heated to 220° C. Upon the addition of the additional water, the temperature of the mixture is reduced to 160° C. The mixture is then stirred at 160° C. for 1.75 h under nitrogen at ambient pressure with a reflux condenser.

After the mixture has reacted for 1.75 h using a reflux condenser, a distillation is performed for 30 minutes in order to remove the excess of water.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

The first phase is filtrated using a filter element having a pore size of about 20 μm.

After the filtration, the first phase includes 74 wt.-% polyol, 16 wt.-% DEG, 6.7 wt.-% aromatic amines, 0.0065 wt.-% potassium and 0.3 wt.-% water.

The second phase includes, directly after the phase separation, less than 5 wt.-% polyol, 67 wt.-% DEG, 14.59 wt. % aromatic amines and 1.5 wt.-% potassium.

During lysis and phase separation according to Example 9, 96% of the polyol and 89% of the amine could be released.

### Example 10

A model polyurethane foam comprising polyol (65 wt.-% of the foam) and toluene diamine (23 wt.-% of the foam) was used as eol polyurethane foam for this Example.

A mixture of 34.7 wt.-% eol polyurethane foam, 60.0 wt.-% diethylene glycol (DEG), 2.0 wt.-% potassium hydroxide as catalyst and 3.3 wt.-% additional water is formed. The additional water is added after the remaining components have been heated to 200° C. Upon the addition of the additional water, the temperature of the mixture is reduced to 170° C. The mixture is then stirred at 170° C. for 1.67 h under nitrogen at ambient pressure with a reflux condenser.

Afterwards, the mixture is cooled to 80° C. and allowed to settle for 16 h while the temperature is kept at about 80° C. After 16 h, a phase separation into a first phase (upper layer, is polyol substance rich) and a second phase (lower layer, alcoholising substance rich) has occurred.

The first phase is filtrated using a filter element having a pore size of about 20 μm.

After the filtration, the first phase includes 78.5 wt.-% polyol, 15 wt.-% DEG, 4.83 wt.-% aromatic amines, 0.0165 wt.-% potassium and 0.5 wt.-% water.

The second phase includes, directly after the phase separation, less than 5 wt.-% polyol, 75 wt.-% DEG, 9.0 wt. % aromatic amines and 1.7 wt.-% potassium.

During lysis and phase separation according to Example 10, 99% of the polyol and 90% of the amine could be released.

### EXAMPLES FOR THE WORK-UP OF THE PHASE, WHICH IS POLYOL SUBSTANCE RICH, PRESENTLY THE FIRST PHASE

### Example 1A1

For Example 1A1, the first phase of Example 1 was used as a starting material.

In order to sediment solids in the first phase, a sedimentation for 24 h is performed. In this respect, the first phase is stored for 24 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 82 wt.-% polyol, 11.1 wt.-% DEG, 3.2 wt.-% aromatic amines and 0.016 wt.-% potassium.

After filtration, the first phase is evaporated in a thin film evaporator at 250° C. and a pressure of 7 mbar followed by an evaporation in a short path evaporator at 250° C. and a pressure of 0.1 mbar.

Afterwards, the first phase includes 99 wt.-% polyol, below 0.1 wt.-% DEG, 0.018 wt.-% aromatic amines, 0.052 wt.-% potassium. The acid number is below 0.01 mg KOH/g.

In a next step, a cation exchange material (5 wt.-% of a strongly acidic resin material having sulfonic groups, here: product having the trade name LEWATIT® K 2621, available from LANXESS Deutschland GmbH) is contacted at 80° C. with the first phase for 24 h in a stirred reactor and filtered using a filter with a pore size of about 70 μm, resulting in a composition of more than 99 wt.-% polyol, below 0.006 wt.-% aromatic amines, below 0.1 wt.-% diethylene glycol and 0.0015 wt.-% potassium. The acid number is 0.12 mg KOH/g.

In a next step, an anion exchange material (2 wt.-% of a strongly basic resin material based on a styrene divinyl benzene copolymer, available under the product name LEWATIT® S 6368 from LANXESS Deutschland GmbH) was contacted at 80° C. with the first phase for 24 h in a stirred reactor and filtered using a filter with a pore size of about 70 μm.

Afterwards, water is removed by a distillation of the first phase at 110° C. at a pressure of 10 mbar, resulting in a composition of more than 99 wt.-% polyol, a DEG content below the limit of detection, below 0.006 wt.-% aromatic amines, 0.0015 wt.-% potassium and an acid number of 0.08 mg KOH/g.

### Example 1A2

For Example 1A2, the first phase of Example 1 was used as a starting material.

In order to sediment solids in the first phase, a sedimentation for 24 h is performed. In this respect, the first phase is stored for 24 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 82 wt.-% polyol, 11.1 wt.-% DEG, 3.2 wt.-% aromatic amines and 0.016 wt.-% potassium.

After filtration, the first phase is evaporated in a thin film evaporator at 250° C. and a pressure of 7 mbar followed by an evaporation in a short path evaporator at 250° C. and a pressure of 0.1 mbar.

Afterwards, the first phase includes 99 wt.-% polyol, below 0.1 wt.-% DEG, 0.018 wt.-% aromatic amines, 0.052 wt.-% potassium. The acid number is below 0.01 mg KOH/g.

In a next step, the first phase is contacted with a 5 wt.-% particulate magnesium silicate (presently a particulate magnesium silicate having the trade name Ambosol MP 20, obtainable from PQ France SAS, 60350 Trosly-Breuil, France), 5 wt.-% activated carbon (presently obtainable under the trade name CNSP 1240) and 2 wt.-% water at 110° C. and 10 mbar for 1 h upon stirring, followed by another 3 hours at full vacuum reaching 4 mbar at the of the 3 hours.

Afterwards, the mixture is filtered using a filter with a pore size of 70 μm, resulting in a composition of more than 99 wt.-% polyol, below 0.1 wt.-% DEG, below 0.008 wt.-% aromatic amines, below 0.0003 wt.-% potassium and an acid number of 0.04 mg KOH/g.

### Example 1A3

For Example 1A3, the first phase of Example 1 was used as a starting material.

In order to sediment solids in the first phase, a sedimentation for 24 h is performed. In this respect, the first phase is stored for 24 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 82 wt.-% polyol, 11.1 wt.-% DEG, 3.2 wt.-% aromatic amines and 0.016 wt.-% potassium.

After filtration, the first phase is evaporated in a thin film evaporator at 250° C. and a pressure of 7 mbar followed by an evaporation in a short path evaporator at 250° C. and a pressure of 0.1 mbar.

Afterwards, the first phase includes 99 wt.-% polyol, below 0.1 wt.-% DEG, 0.018 wt.-% aromatic amines, 0.052 wt.-% potassium. The acid number is below 0.01 mg KOH/g.

In a next step, the first phase is contacted with a 5 wt.-% particulate magnesium silicate (presently a particulate magnesium silicate having the trade name Ambosol MP 20, obtainable from PQ France SAS, 60350 Trosly-Breuil, France), and 2 wt.-% water at 80° C. for 1 h while stirring and filtered using a filter having a pore size of about 70 μm.

Afterwards, water is removed by a distillation at 110° C. and under a pressure of 10 mbar, resulting in a composition of more than 99 wt.-% polyol, below 0.1 wt.-% DEG, below 0.02 wt.-% aromatic amines, below 0.0003 wt.-% potassium and an acid number of 0.04 mg KOH/g.

The comparison of Example 1A2 and Example 1A3 shows that with a combination of magnesium silicate and activated carbon drastically more aromatic amines can be removed than with magnesium silicate alone.

### Example 2A1

For Example 2A1, the first phase of Example 2 was used as a starting material.

In order to sediment solids in the first phase, a sedimentation for 10 h is performed. In this respect, the first phase is stored for 10 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 79 wt.-% polyol, 10.7 wt.-% DEG, 3.5 wt.-% aromatic amines and 0.025 wt.-% potassium.

After filtration, the first phase is evaporated in a thin film evaporator at 200° C. and a pressure of 7 mbar followed by an evaporation in a short path evaporator at 200° C. and a pressure of 7 mbar.

Afterwards, the first phase includes 99 wt.-% polyol, 0.21 wt.-% DEG, 0.25 wt.-% aromatic amines and 0.045 wt.-% potassium. The acid number is below 0.01 mg KOH/g.

In a next step, a cation exchange material (9 wt.-% of a strongly acidic resin material having sulfonic groups, here: product having the trade name LEWATIT® K 2629, available from LANXESS Deutschland GmbH) is contacted at 80° C. with the first phase for 24 h in a stirred reactor and filtered using a filter having a pore size of about 70 μm, resulting in a composition of more than 99 wt.-% polyol, 0.21 wt.-% DEG, below 0.006 wt.-% aromatic amines, and 0.0010 wt.-% potassium. The acid number is 0.63 mg KOH/g.

Afterwards, the first phase is contacted with an anion exchange material in the form of a weakly basic, macroporous anion exchange resin with tertiary amine groups (monofunctional) (obtainable under the trade name LEWATIT® MP 62 (available from LANXESS Deutschland GmbH). A mixture of the first phase and 18 wt.-% of the anion exchange material is prepared, stirred at 80° C. for 24 h and filtered using a filter having a pore size of about 70 μm. This step of contacting the first phase with the anion exchange material is performed twice (using fresh anion exchange material for each repetition).

Afterwards, the first phase is distilled to remove water at a temperature of 110° C. using a pressure of 10 mbar, resulting in a composition including 99 wt.-% polyol, 0.13 wt.-% DEG, below 0.006 wt.-% aromatic amines and 0.001 wt.-% potassium. The acid number is 0.04 mg KOH/g.

### Example 2A2

For Example 2A2, the first phase of Example 2 was used as a starting material.

In order to sediment solids in the first phase, a sedimentation for 10 h is performed. In this respect, the first phase is stored for 10 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 79 wt.-% polyol, 10.7 wt.-% DEG, 3.5 wt.-% aromatic amines and 0.025 wt.-% potassium.

After filtration, the first phase is evaporated in a thin film evaporator at 200° C. and a pressure of 7 mbar followed by an evaporation in a short path evaporator at 200° C. and a pressure of 7 mbar.

Afterwards, the first phase includes 99 wt.-% polyol, 0.21 wt.-% DEG, 0.25 wt.-% aromatic amines and 0.045 wt.-% potassium. The acid number is below 0.01 mg KOH/g.

In a next step, the first phase is contacted with an activated carbon material. In this regard, a mixture of the first phase and 5 wt.-% activated carbon (presently obtainable under the trade name CNSP 1240) is prepared. The mixture is stirred at 80° C. for 1 hour and filtered using a filter having a pore size of about 70 μm. Afterwards, water is removed by performing a distillation at 110° C. and 10 mbar, resulting in a composition including 99 wt.-% polyol, 0.21 wt.-% DEG, less than 0.0075 wt.-% aromatic amines and 0.0039 wt.-% potassium. The acid number is 0.09 mg KOH/g.

Example 2A2 compared to Example 1A2 illustrates that magnesium silicate appears to be more efficient regarding the potassium removal compared to activated carbon alone.

### Example 3A1

For Example 3A1, the first phase of Example 3 was used as a starting material.

In order to sediment solids in the first phase, a sedimentation for 24 h is performed. In this respect, the first phase is stored for 24 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 80 wt.-% polyol, 11.5 wt.-% DEG, 3.3 wt.-% aromatic amines and 0.023 wt.-% potassium.

After filtration, the first phase is evaporated in a thin film evaporator at 200° C. and a pressure of 1 mbar.

Afterwards, the first phase includes 99 wt.-% polyol, 0.21 wt.-% DEG, 0.13 wt.-% aromatic amines, 0.025 wt.-% potassium. The acid number is below 0.01 mg KOH/g.

In a next step, the first phase is contacted with a cation exchange material in the form of a strongly acidic resin material having sulfonic groups (here: product having the trade name LEWATIT® K 2621, available from LANXESS Deutschland GmbH) and an anion exchange material in the form of a weakly basic, macroporous anion exchange resin with tertiary amine groups (monofunctional) (obtainable under the trade name LEWATIT® MP 62 (available from LANXESS Deutschland GmbH). A mixture of the first phase, 9 wt.-% cation exchange material and 9 wt.-% anion exchange material is stirred for 1 hour at 80° C. and filtered using a filter having a pore size of about 70 μm.

Afterwards, the first phase is distilled at 200° C. under a pressure of 100 mbar in order to remove water, resulting in a composition including 99 wt.-% polyol, 0.12 wt.-% DEG, below 0.006 wt.-% aromatic amines and 0.0009 wt.-% potassium. The acid number is 0.04 mg KOH/g.

Example 3A1 illustrates that cation exchange material and anion exchange material can be used simultaneously.

### Example 4A1

For Example 4A1, the first phase of Example 4.1 was used as a starting material.

In order to sediment solids in the first phase, a sedimentation for 24 h is performed. In this respect, the first phase is stored for 24 h in a storage tank.

The supernatant is then filtrated using a cascade of filters, wherein the first filter has a pore size of 270 μm, the second filter has a pore size of 70 μm and the third filter has a pore size of 20 μm.

After the filtration, the first phase includes 83 wt.-% polyol, 12.4 wt.-% DEG, 3.1 wt.-% aromatic amines and 0.025 wt.-% potassium.

After filtration, the first phase is evaporated in a thin film evaporator at 250° C. and a pressure of 6.5 mbar followed by an evaporation in a short path evaporator at 250° C. and a pressure of 0.15 mbar.

Afterwards, the first phase includes 99 wt.-% polyol, less than 0.1 wt.-% DEG, 0.02 wt.-% aromatic amines and 0.032 wt.-% potassium. The acid number is 0.01 mg KOH/g.

In a next step, the first phase is contacted with mixture of 2 wt.-% particulate magnesium silicate (presently a particulate magnesium silicate having the trade name Ambosol MP 20, obtainable from PQ France SAS, 60350 Trosly-Breuil, France), 3 wt.-% activated carbon (presently obtainable under the trade name CNSP 1240) and 2 wt.-% water at 110° C. and 10 mbar for 1 h upon stirring, followed by another 3 hours at full vacuum reaching 4 mbar at the of the 3 hours.

Afterwards, the mixture is filtered using a filter with a pore size of 70 μm, resulting in a composition of more than 99 wt.-% polyol, below 0.1 wt.-% DEG, below 0.001 wt.-% aromatic amines, below 0.0003 wt.-% potassium and an acid number of 0.03 mg KOH/g.

### Example 5A1

For Example 5A1, the first phase of Example 5 was used as a starting material.

The first phase is filtered with a filter of a pore size of 90 μm.

Afterwards the first phase is contacted with magnesium silicate (presently a particulate magnesium silicate having the trade name Ambosol MP 20, obtainable from PQ France SAS, 60350 Trosly-Breuil, France) by preparing a mixture of the first phase and 3 wt.-% magnesium silicate. The mixture is stirred at 100° C. for 2.5 hour.

Afterwards (without further sedimentation) the first phase is filtrated using a filter having a pore size of about 3 μm.

After the filtration, the first phase includes 81 wt.-% polyol, 13.1 wt.-% DEG, 5.7 wt.-% aromatic amines and 0.0043 wt.-% potassium.

After filtration, the first phase is evaporated in a flash evaporator at 190° C. and a pressure of 30 mbar followed by an evaporation in a short path evaporator at 250° C. and a pressure of 0.15 mbar.

Afterwards, the first phase includes 99 wt.-% polyol, less than 0.1 wt.-% DEG, less than 0.005 wt.-% aromatic amines and 0.0054 wt.-% potassium. The acid number is below 0.1 mg KOH/g.

### Example 7A1

For Example 7A1, the first phase of Example 7 was used as a starting material.

Without sedimentation, the first phase is filtrated using a filter having a pore size of 20 μm, resulting in a composition including 59 wt.-% polyol, 19 wt.-% DEG, 8.4 wt.-% aromatic amines, 0.044 wt.-% potassium and 0.4 wt. % water.

No further work-up is performed as up to this point it is already clear that a polyurethane material having a SAN content of up to 15 wt. % can be processed.

### Example 8A1

For Example 8A1, the first phase of Example 8 was used as a starting material.

Without sedimentation, the first phase is filtrated using a filter having a pore size of 20 μm, resulting in a composition including 79 wt.-% polyol, 16 wt.-% DEG, 3.98 wt.-% aromatic amines, 0.0009 wt.-% potassium and 0.3 wt. % water.

No further work-up is performed.

### Example 9A1

For Example 9A1, the first phase of Example 9 was used as a starting material.

Without sedimentation, the first phase is filtrated using a filter having a pore size of 20 μm, resulting in a composition including 74 wt.-% polyol, 16 wt.-% DEG, 6.7 wt.-% aromatic amines, 0.0067 wt.-% potassium and 0.3 wt. % water.

No further work-up is performed.

### Example 10A1

For Example 10A1, the first phase of Example 10 was used as a starting material.

Without sedimentation, the first phase is filtrated using a filter having a pore size of 20 μm, resulting in a composition including 78.5 wt.-% polyol, 15 wt.-% DEG, 4.83 wt.-% aromatic amines, 0.0165 wt.-% potassium and 0.5 wt. % water.

No further work-up is performed.

Examples for the work-up of the phase, which is amine substance rich, presently the second phase:

### Example 1B1

No further work-up of the second phase is performed.

### Example 2B1

The second phase resulting after the phase separation is mixed with the distillate of the first phase, resulting from Example 2A1. This combined phase as a whole is subsequently referred to as second phase.

The second phase is distilled in a first distillation comprising two parts.

In a first part of the first distillation, DEG is removed by performing a distillation with a sump temperature of 230° C., a head temperature of 170° C. and a head pressure of 350 mbar.

Afterwards, a second stage of the first distillation is performed in which the TDA is distilled. The second stage of the first distillation is performed using a sump temperature of 220° C., a head temperature of 130° C. and a head pressure of 4 mbar.

### SIMULATIONS

### Example 11

A stream (above referred to as second phase 112) consisting of diethylene glycol (DEG), a mixture of toluene diamine (TDA) isomers, water, by-products from a hydroglycolysis reaction (e.g. potassium hydroxide, potassium carbonate and potassium hydrogen carbonate) and impurities originating from the polyurethane material 100 is treated in the following process:
1) filtration to remove solids from the stream (second phase 112);
2) a first distillation stage 136a in a sump distillation column to remove DEG and water; and
3) a second distillation stage 136b to remove residual DEG from the TDA resulting in a crude TDA stream;
4) the crude TDA stream, comprising 2,4- and 2,6 TDA, is afterwards fed into a TDA purification distillation column 140 of an existing TDA plant.

The above-described process is simulated with Chemasim™ simulation (version 6.6, stationary process simulation software) and using the following parameters:

From a stream (second phase 112) having the following composition:

$$m ( DEG ) = 52.5 kg / h$$

$$m ( 2 , 4 - TDA ) = 24. kg / h$$

$$m ( 2 , 6 - TDA ) = 6. kg / h$$

$$m ( {H}_{2} O ) = 2.5 kg / h$$

$$m ( solids ) = 15. kg / h$$

15.0 kg/h solids are removed in a filtration at 80° C. The liquid filtrate (m=85.0 kg/h) is fed into a first distillation stage 136a (distillation column, p=100 mbar, reflux ratio=4 g/g, number of theoretical stages: 18) at the evaporator stage. 71 kW of energy are supplied to the evaporator resulting in a sump temperature to 198.5° C. Due to the energy supplied, the liquid is brought to the boil. A head stream with Tgas=78° C. having the composition:

$$w ( DEG ) = 0.952 g / g$$

$$w ( 2 , 4 - TDA = 219 ppb$$

$$w ( 2 , 6 - TDA ) = 32 ppb$$

$$w ( {H}_{2} O ) = 0.048 g / g$$

and a sump stream with T_{liquid}=198.5° C. having the composition

$$w ( DEG ) = 0.077 g / g$$

$$w ( 2 , 4 - TDA = 0.738 g / g$$

$$w ( 2 , 6 - TDA ) = 0.185 g / g$$

$$w ( {H}_{2} O ) = 23 ppm$$

are formed.

The head stream is condensed and recycled as liquid to the reaction section. The sump stream is transferred to a second distillation stage 136b (distillation column, p=70 mbar, reflux ratio=4 g/g, number of theoretical stages: 20) for further purification. Solids, that might precipitate in the distillation sump during the removal of DEG and water are removed using a filter that is integrated into the external circulation loop of the evaporator stage.

The sump stream is fed to the second distillation stage 136b at the level of the theoretical stage 10. 4.1 kW of energy are supplied to the evaporator in the sump of the column, causing the liquid to boil. A head stream with T_{gas}=170.6° C. having the composition

$$w ( DEG ) = 0.484 g / g$$

$$w ( 2 , 4 - TDA = 0.43 g / g$$

$$w ( 2 , 6 - TDA ) = 0.086 g / g$$

$$w ( H2O ) = 148 ppm$$

and a sump stream with T_{liquid}=194.2° C. having the composition

$$w ( DEG ) = 300 ppm$$

$$w ( 2 , 4 - TDA = 0.797 g / g$$

$$w ( 2 , 6 - TDA ) = 0.203 g / g$$

$$w ( {H}_{2} O ) = 0 g / g$$

are formed.

The resulting crude TDA stream can be further purified in a third distillation 136c (third distillation column) to remove traces of DEG. This third distillation stage 136c can either be a standalone TDA distillation purifying TDA by removing the TDA isomers as side-stream, traces of DEG as head stream and impurities with boiling points above the TDA isomers as TDA TAR or an existing TDA purification column 140 in an existing TDA producing plant.

### Example 12

A stream (second phase 112), consisting of diethylene glycol (DEG), a mixture of TDA isomers, water, by-products from a hydroglycolysis reaction (e.g., potassium hydroxide, potassium carbonate and potassium hydrogen carbonate) and impurities originating from the polyurethane material 100 is treated in the following process:
1) filtration to remove solids from the stream (second phase 112);
2) TDA extraction and recovery of the solvent used for the extraction (extraction solvent in the form of, e.g., toluene, xylene, di-isopropyl ether), comprising extraction of TDA by means of the extraction solvent, recovery of the extraction solvent and purification of the TDA stream in a first distillation stage 136a;
3) distillation of the TDA stream in a second distillation stage 136b to remove residual DEG resulting in a TDA stream, also referred to as crude TDA, comprising different TDA isomers (2,4-und 2,6-TDA); and
4) purification of the crude TDA in a third distillation stage 136c to purify the TDA. This third distillation stage 136c can either be done in a dedicated distillation stage or in a distillation already existing as part of an existing TDA producing plant.

The above-described process is simulated with Chemasim™ simulation (version 6.6, stationary process simulation software) and using the following parameters:

From a stream (second phase 112) having the following composition:

$$m ( DEG ) = 52.5 kg / h$$

$$m ( 2 , 4 - TDA ) = 24. kg / h$$

$$m ( 2 , 6 - TDA ) = 6. kg / h$$

$$m ( {H}_{2} O ) = 2.5 kg / h$$

$$m ( solids ) = 15. kg / h$$

15.0 kg/h solids are removed in a filtration at 80° C. resulting in a liquid filtrate. To the liquid filtrate (m=85.0 kg/h) water is added to improve the extraction with a non-polar extraction solvent. In a specific example, 30 kg/h water is added, resulting in a mixture having the following composition:

$$w ( DEG ) = 0.457 g / g$$

$$w ( 2 , 4 - TDA ) = 0.209 g / g$$

$$w ( 2 , 6 - TDA ) = 0.052 g / g$$

$$w ( {H}_{2} O ) = 0.283 g / g .$$

In an extraction column (T=50° C., p=1 bar, stages: 10) operated in countercurrent mode 200 kg/h toluene are used to obtain an extract stream having the following composition:

$$w ( DEG ) = 0.01 g / g$$

$$w ( 2 , 4 - TDA ) = 0.103 g / g$$

$$w ( 2 , 6 - TDA ) = 0.024 g / g$$

$$w ( {H}_{2} O ) = 0.003 g / g$$

$$w ( Toluol ) = 0.86 g / g$$

and a raffinate stream having the following composition:

$$w ( DEG ) = 0.603 g / g$$

$$w ( 2 , 4 - TDA ) = 5.9 * {10}^{- 7} g / g$$

$$w ( 2 , 6 - TDA ) = 0.006 g / g$$

$$w ( {H}_{2} O ) = 0.384 g / g$$

$$w ( Toluol ) = 0.007 g / g .$$

The extract stream is purified in a subsequent first distillation stage 136a. The distillation is done at 0.2 bar, a head temperature of 49° C. and a sump temperature of 225.6° C. A reflux ratio 0.5 g/g is used. The column has 20 theoretical stages. A sump stream containing

$$w ( DEG ) = 3.4 * {10}^{- 5} g / g$$

$$w ( 2 , 4 - TDA ) = 0.814 g / g$$

$$w ( 2 , 6 - TDA ) = 0.186 g / g$$

$$w ( {H}_{2} O ) = 0 g / g$$

$$w ( Toluol ) = 0 g / g$$

is obtained.

This stream can be further purified using an overhead distillation column or a side-stream distillation column to remove the high boiling impurities as TDA TAR in a TDA-purification column as described in the prior art. This TDA distillation column (also referred to as third distillation stage 136c) can be either a dedicated distillation column or a distillation column 140 integrated into an existing TDA producing plant.

The head stream of the first distillation stage 136a, consisting of

$$w ( DEG ) = 0.012 g / g$$

$$w ( 2 , 4 - TDA ) = 4.2 * 10 - 8 g / g$$

$$w ( 2 , 6 - TDA ) = 5.5 * 10 - 9 g / g$$

$$w ( {H}_{2} O ) = 0 \text{.003} g / g$$

$$w ( Toluol ) = 0.985 g / g$$

is transferred to a phase separator where a DEG-rich and a toluene-rich layer are formed. The DEG-rich layer having

$$w ( DEG ) = 0.708 g / g$$

$$w ( 2 , 4 - TDA ) = 33 ppb$$

$$w ( 2 , 6 - TDA ) = 11 ppb$$

$$w ( {H}_{2} O ) = 0 \text{.280} g / g$$

$$w ( Toluol ) = 0.012 g / g$$

is recycled to the reaction section, the toluene-rich layer

$$w ( DEG ) = 0.006 g / g$$

$$w ( 2 , 4 - TDA ) = 42 ppb$$

$$w ( 2 , 6 - TDA ) = 5 ppb$$

$$w ( {H}_{2} O ) = 0 \text{.001} g / g$$

$$w ( Toluol ) = 0.993 g / g$$

is re-used in the (TDA-)extraction column.

The raffinate stream of the extraction column is transferred to a distillation column, wherein DEG and water are distilled off as head stream and the solid impurities of the stream (second phase 112) are purged. The head stream is, after condensation transferred to the phase separation step, separating the toluene-rich and the DEG-rich layer. From there it is recycled back to the solvolysis reaction.

### Example 13

In a laboratory-scale distillation setup consisting of a distillation column as described in Table 1, the second phase 112 obtained from a hydroglycolysis of a polyurethane material 100 is distilled.

TABLE 1
Design and operating conditions laboratory-
scale distillation column:

| Packing | BX (simulated) | |
|---|---|---|
| Packing height | 140 | cm |
| Theoretical stages | 20 | |
| Head pressure | >2 | mbar |
| Δp | 2 mbar to 15 mbar | |
| Outer diameter | 9 cm/5 cm | |
| Column height | 150 | cm |

| | |
|---|---|
| Column material | glass |
| Evaporator | Natural circulation |
| Condenser | glass (oil as |
| | cooling medium) |

The stream (second phase 112) was composed of:

$$m ( DEG ) = 1396.4 g$$

$$m ( TDA ) = 207.5 g$$

$$m ( MDA ) = 10.1 g$$

$$m ( salts ) = 51.3 g$$

$$m ( {H}_{2} O ) = 9.7 g$$

$$m ( unknowns ) = 243.1 g$$

The used column was operated in batch mode. Fractions of 20 to 100 g were removed via the head of the column and analyzed using HPLC. Detailed information on the removed amounts and corresponding operating conditions are summarized in Table 2.

TABLE 2
Overview over fractions and operating conditions of the fractionating column

| Fraction | m | m | T | T | p |
|---|---|---|---|---|---|
| # | (fraction)/g | (accumulated.)/g | (sump)/° C. | (head)/° C. | (head)/mbar |
| 1 | 61.96 | 61.96 | 132 | 114 | 6.7 |
| 2 | 70.82 | 132.78 | 133 | 118 | 8.0 |
| 3 | 64.76 | 197.54 | 133 | 116.8 | 7.7 |
| 4 | 57.34 | 254.88 | 134 | 117.4 | 7.9 |
| 5 | 74.24 | 329.12 | 134 | 117.7 | 8.0 |
| 6 | 73.64 | 402.76 | 134 | 118.1 | 8.3 |
| 7 | 80.97 | 483.73 | 135 | 118.9 | 8.6 |
| 8 | 60.42 | 544.15 | 136 | 117 | 8.0 |
| 9 | 58.61 | 602.76 | 136 | 117.6 | 8.0 |
| 10 | 69.63 | 672.39 | 137 | 118.3 | 8.5 |
| 11 | 66.16 | 738.55 | 140 | 118.5 | 8.8 |
| 12 | 80.77 | 819.32 | 142 | 117.8 | 8.0 |
| 13 | 79.04 | 898.36 | 147 | 116.7 | 7.7 |
| 14 | 72.73 | 971.09 | 145 | 118.3 | 8.3 |
| 15 | 67.51 | 1038.6 | 141 | 118.2 | 8.6 |
| 16 | 73 | 1111.6 | 144 | 116.6 | 7.7 |
| 17 | 75.34 | 1186.94 | 150 | 117.3 | 8 |
| 18 | 65.51 | 1252.45 | 151 | 118.8 | 8 |
| 19 | 34.48 | 1286.93 | 152 | 119 | 8.6 |
| 20 | 20.47 | 1307.4 | 151 | 125 | 8.3 |
| 21 | 33.24 | 1340.64 | 157 | 138.2 | 8 |
| 22 | 41.32 | 1381.96 | 158 | 138.6 | 8.2 |

Fractions no. 1 to 17 are pure DEG. Starting from fraction no. 18, TDA can be drawn off via the head of the column. With increasing sump temperature (and distillation time, respectively) the TDA content of the fraction drawn off via the head of the column increases. As off fraction no. 20, TDA dominates (fraction no. 20 contains 52 wt.-% TDA). After a mixed fraction (no. 22) containing minor amounts of DEG, a TDA fraction can be obtained.

1.-16. (canceled)

17. A process for recovering an amine substance from a polyurethane material wherein the process comprises the following:
alcoholising a polyurethane material by contacting the polyurethane material with an alcoholising substance, wherein water is added in an amount so that a water content of a resulting mixture is from about 0.2 eq. to about 30 eq., based on the amount of cleavable bonds of the polyurethane material, wherein during alcoholising a mixture containing a polyol substance and an amine substance is formed;
allowing the mixture to settle, wherein a phase which is polyol substance rich, and a phase which is alcoholising substance rich, are formed;
work-up of the mixture by purification of the amine substance, including the following:
distilling the phase, which is alcoholising substance rich in a first distillation, in order to purify the amine substance, wherein the first distillation includes one or more distillation stages.

18. The process according to claim 17, wherein the amine substance comprises or consists of a toluene diamine (TDA)-based substance.

19. The process according to claim 17, wherein an excess of water is removed before allowing the mixture to settle by heating the mixture and/or applying a vacuum.

20. The process according to claim 17, wherein an inner temperature of the mixture is kept at about 150° C. to about 240° C. and at a pressure of about 1 bar to about 60 bar, during and after water addition.

21. The process according to claim 17, wherein the first distillation is performed at a temperature of about 130° C. to about 290° C. and at a pressure of about 1 mbar to about 1000 mbar and/or wherein the first distillation (136) is performed in one or more distillation columns.

22. The process according to claim 17, wherein the first distillation comprises a first distillation stage, in which the alcoholising substance is removed by distillation, and a second distillation stage, in which the amine substance is purified by distillation.

23. The process according to claim 22, wherein the first distillation stage is performed at a temperature of about 130° C. to about 250° C. and at a pressure of about 10 mbar to about 1000 mbar and/or the second distillation stage is performed at a temperature of about 140° C. to about 250° C. and at a pressure of about 1 mbar to about 200 mbar.

24. The process according to claim 17, wherein the first distillation comprises a first distillation stage, a second distillation stage and a third distillation stage, wherein the first distillation stage is performed at a temperature of about 60° C. to about 270° C. and at a pressure of about 100 mbar to about 1000 mbar and/or the second distillation stage is performed at a temperature of about 130° C. to about 250° C. and at a pressure of about 10 mbar to about 500 mbar and/or the third distillation stage is performed at a temperature of about 140° C. to about 250° C. and at a pressure of about 1 mbar to about 200 mbar.

25. The process according to claim 17, wherein the alcoholising step is performed so that the mixture separates into the first phase and the second phase during settling by adjusting a temperature of the mixture to about 25° C. to about 160° C. and by keeping the mixture at this temperature until phase separation has occurred.

26. The process according to claim 17, wherein the phase, which is polyol substance rich is distilled in a second distillation, wherein a second distillate is obtained, wherein preferably the second distillate is combined with the phase, which is alcoholising substance rich before the first distillation.

27. The process according to claim 17, wherein the phase, which is the alcoholising substance rich is treated in a solid-liquid-separation before the first distillation is performed by one or more of the following: filtration, centrifugation, decantation, extraction.

28. The process according to claim 17, wherein after the work-up of the phase, which is alcoholising substance rich a yield of the amine substance release is 70% or higher determined by the actually obtained amount divided by the theoretically obtainable amount.

29. The process according to claim 17, wherein no hydrolysis of the phase, which is alcoholising substance rich is performed.

30. The process according to claim 17, wherein the polyurethane material (100) is a polyurethane foam, a polyurethane elastomer, a polyurethane adhesive, a polyurethane coating or a mixture thereof.

31. The process according to claim 17, wherein at least a final distillation stage of the first distillation (136) is performed within an amine plant.

32. A process for producing an isocyanate substance from an amine substance obtained by a process according to claim 17.
